(19) United States
(12) Patent Application Publication
(10) Pub. No.: US 2021/0361875 A1
(43) Pub. Date: Nov. 25, 2021

(54) Surface Texturization for Rolling Diaphragm Syringe

(71) Applicant: BAYER HEALTHCARE LLC, WHIPPANY, NJ (US)

(72) Inventors: Kevin Cowan, Allison Park, PA (US); Michael Spohn, Fenelton, PA (US); Michael Swantner, Saxonburg, PA (US); James Mierzwiak, Monroe, MI (US); David Berry, Kittanning, PA (US); Barry Tucker, Verona, PA (US); Arthur Uber, III, Pittsburgh, PA (US)

(21) Appl. No.: 16/965,865

(22) PCT Filed: Feb. 5, 2019
(86) PCT No.: PCT/US2019/016621

§ 371 Date: Jul. 29, 2020

Related U.S. Application Data
(60) Provisional application No. 62/626,400, filed on Feb. 5, 2018.

(51) Int. Cl. A61M 5/315

(52) U.S. Cl. CPC A61M 5/31513; A61M 5/31501; A61M 2207/00; A61M 2205/0222; A61M 2005/3151

(57) ABSTRACT

A rolling diaphragm syringe for receiving a medical fluid therein, the rolling diaphragm syringe comprising a sidewall that is flexible and rolls upon itself when acted upon by the piston such that an outer surface of the sidewall is rolled in a radially inward direction as the piston is advanced from the proximal end to the distal end, and wherein at least a portion of an inner surface of the flexible sidewall comprises at least one surface texturization feature.

### CROSS REFERENCE TO RELATED APPLICATIONS

The present application is a § U.S. national phase application under 35 U.S.C. § 371 of PCT International Application No. PCT/US2019/016621, filed 5 Feb. 2019 and claims priority to U.S. Provisional Application No. 62/626,400, filed 5 Feb. 2018, the disclosures of which are incorporated herein in their entirety.

### BACKGROUND OF THE DISCLOSURE

### Field of the Disclosure

The present disclosure is related to containers and syringes for use in the medical field and, more particularly, to a rolling diaphragm-type container and/or syringe having a flexible sidewall with a texturized inner surface that rolls upon itself when acted upon by a piston for selectively filling the syringe with a fluid and discharging the fluid from the syringe. The present disclosure is also directed to methods, features, and apparatus for texturizing the inner surface of the flexible sidewall and/or an injection molded preform.

### Description of Related Art

In many medical diagnostic and therapeutic procedures, a medical practitioner, such as a physician, injects a patient with one or more medical fluids. In recent years, a number of injector-actuated syringes and powered fluid injectors for pressurized injection of medical fluids, such as a solution of contrast media for imaging procedures (often referred to simply as “contrast”), a flushing agent, such as saline, and other medical fluids, have been developed for use in procedures such as angiography, computed tomography (CT), ultrasound, magnetic resonance imaging (MRI), positron emission tomography (PET), and other imaging procedures. In general, these fluid injectors are designed to accurately deliver preset amounts of fluid at a preset pressure and/or flow rate.

Typically, powered injectors have one or more drive members, such as pistons, that connect to a syringe plunger within a syringe. The syringe generally includes a rigid barrel with the syringe plunger being slidably disposed within the barrel. The drive members drive the plungers in a proximal and/or distal direction along a longitudinal axis of the barrel to draw fluid into the syringe barrel or deliver the fluid from the syringe barrel, respectively.

Many syringes used in the medical field are typically disposable and are discarded after one use or a limited number of uses. Although disposable syringes are typically made by mass production methods such as injection molding, these disposable syringes may be relatively expensive due to the overall number of imaging procedures performed at an imaging facility, the materials, precision, and volume and weigh of raw materials involved in their manufacture, and economic costs associated with packaging, shipping (volume and weight), storage, and disposal costs associated with conventional syringes. Accordingly, it remains desirable to develop improved designs of syringes to facilitate injection procedures with lower associated costs.

### SUMMARY OF THE DISCLOSURE

The present disclosure generally relates to rolling diaphragm containers, such as rolling diaphragm syringes having a flexible sidewall that rolls upon itself when acted upon by a piston such that an outer surface of the sidewall rolls in a radially inward direction as the piston is advanced from a proximal end to a distal end of the rolling diaphragm syringe and unrolls in a radially outward direction as the piston is retracted from the distal end to the proximal end, wherein at least a portion of an inner surface of the flexible sidewall comprises at least one surface texturization feature. According to various embodiments, the at least one surface texturization feature may result in at least one of a reduction the coefficient of friction, such as for surface to surface kinetic friction, between two portions of an inner surface of flexible sidewall of a rolling diaphragm syringe as the surfaces move relative to each other, a reduction of audible noise as the two portions of the inner surface move relative to each other, and a decrease in the volume of air entrapped between the rolled inner surfaces of the rolling diaphragm syringe. One further benefit is the reduction of particulate generation during the rolling/unrolling processes due to the reduction of the frictional forces between the two portions of the inner surface as they slide over or move relative to one another.

According to a first embodiment, the present disclosure provides a rolling diaphragm syringe for receiving a medical fluid therein. The rolling diaphragm syringe may comprise a closed proximal end wall for releasably engaging a piston of a fluid injector, a distal end having a neck and a fluid outlet, a flexible sidewall extending between the proximal end wall and the distal end, wherein the flexible sidewall rolls upon itself when acted upon by the piston such that the outer surface of the flexible sidewall rolls in a radially inward direction as the piston is advanced from the proximal end to the distal end and unrolls in a radially outward direction as the piston is retracted from the distal end to the proximal end, and at least one surface texturization feature on at least a portion of an inner surface of the flexible sidewall.

The rolling diaphragm syringe may be made from a medical grade polymer, such as, a medical grade polyethylene terephthalate (PET). The at least one surface texturization feature may reduce the coefficient of friction (μ) between contacting portions of the inner surface of the flexible sidewall as it is rolled and/or unrolled and the contacting portions move relative to one another. In certain embodiments, the coefficient of friction (μ) between the contacting portions of the inner surface of the flexible sidewall having the at least one surface texturization feature may range from 0.10 to 2.30 as the flexible sidewall is rolled or unrolled. In various embodiments, the at least one surface texturization feature may maintain a fluid pathway between parallel inner surfaces of the at least partially rolled rolling diaphragm syringe to allow air to escape from between the parallel inner surfaces during a rolling or unrolling process.

The at least one surface texturization feature may be selected from a group of texture pattern consisting of a plurality of uniform or non-uniform longitudinal ribs, a plurality of uniform or non-uniform ribs having a spiral configuration around a circumference of the inner surface, a plurality of ribs having a non-uniform pattern on the inner surface, a plurality of flat surfaces, a uniform or non-uniform roughened surface, a plurality of dimpled protrusions extending from the inner surface of the flexible sidewall, a plurality of particulates or beads embedded in the sidewall, or any combinations thereof.

According to various aspects, the at least one surface texturization feature may be a plurality of longitudinal ribs. The plurality of longitudinal ribs may be uniformly or non-uniformly arranged around the inner surface of at least the flexible sidewall. In certain embodiments, the plurality of longitudinal ribs may extend to at least a portion of the proximal end wall and/or the distal conical portion of the rolling diaphragm. In other embodiments, the plurality of longitudinal ribs may extend only partially from the proximal end to the distal end or only partially from the distal end to the proximal end. In certain embodiments, at least a portion of the plurality of longitudinal ribs may extend for different lengths and/or at different regions along the longitudinal axis along the inner surface of the flexible sidewall of the rolling diaphragm syringe. In other embodiments, the plurality of longitudinal ribs may have different heights and/or widths along the lengths of the longitudinal axis of the longitudinal rib.

According to other embodiments, the at least one surface texturization feature may be a plurality of uniform or non-uniform ribs having a spiral configuration around a circumference of the inner surface of the flexible sidewall extending along at least a portion of the longitudinal axis. In certain embodiments, the plurality of spiral ribs may extend to at least a portion of the proximal end wall and/or the distal conical portion of the rolling diaphragm. In other embodiments, the plurality of spiral ribs may extend only partially from the proximal end to the distal end or only partially from the distal end to the proximal end. In certain embodiments, at least a portion of the plurality of spiral ribs may extend for different lengths and/or at different regions along the circumference and/or longitudinal axis of the inner surface of the flexible sidewall of the rolling diaphragm syringe. In other embodiments, the plurality of spiral ribs may have different heights and/or widths along the circumference and/or longitudinal axis of the inner surface of the flexible sidewall.

Other embodiments of the present disclosure may be directed to a preform for blow-molding, such as stretch blow-molding, a rolling diaphragm syringe. The preform may be made from an injection molding process and may be made of any suitable medical grade plastic. The preform may comprise a closed proximal end portion having a piston engagement feature configured for allowing releasable engagement between the rolling diaphragm syringe and a piston of a fluid injector, a distal end having a fluid outlet, a sidewall having an inner surface and an outer surface, and at least one preform texturization feature on at least a portion of one of the inner surface and/or the outer surface of the sidewall, wherein the at least one preform texturization feature forms an at least one surface texturization feature on at least a portion of an inner surface of a sidewall of the rolling diaphragm syringe, for example during the blow-molding process. The at least one preform texturization feature may comprise a plurality of preform longitudinal ribs on at least a portion of the inner surface of the preform sidewall, wherein the plurality of preform longitudinal ribs form a plurality of longitudinal ribs or a plurality of spiral ribs on at least a portion of the inner surface of the sidewall of the rolling diaphragm syringe. In certain embodiments, the at least one preform texturization feature may comprise a plurality of preform longitudinal ribs on at least a portion of the outer surface of the sidewall, wherein the plurality of preform longitudinal ribs form a plurality of longitudinal ribs or a plurality of spiral ribs on at least a portion of the outer surface of the sidewall of the rolling diaphragm syringe, and wherein the plurality of longitudinal ribs or the plurality of spiral ribs on at least a portion of the outer surface of the sidewall of the rolling diaphragm syringe are converted to a plurality of longitudinal ribs or a plurality of spiral ribs on at least a portion of the inner surface of the sidewall of the rolling diaphragm syringe during a blow-molding process.

According to various embodiments, the at least one preform texturization feature on at least the portion of one of the inner surface and/or the outer surface of the preform sidewall may be formed during an injection molding process using a mold having corresponding grooves on the core structure of the injection mold or the outer mold cavity of the injection mold. In certain embodiments, the at least one preform texturization feature may comprise a plurality of preform longitudinal ribs on at least a portion of the inner surface of the preform sidewall, wherein the plurality of preform longitudinal ribs are formed by etching or cutting a plurality of longitudinal grooves on the at least a portion of the inner surface of the sidewall during removing of an injection mold core structure having a corresponding plurality of groove etching or cutting members.

Still other embodiments of the present disclosure may be directed to methods for reducing friction, for example by reducing a coefficient of friction (μ), between the contacting portions of a rolled inner surface of the flexible sidewall of a rolling diaphragm syringe during a rolling and/or unrolling process. The methods may include texturizing at least a portion of the inner surface of the flexible sidewall with at least one surface texturization feature selected from the group consisting of a plurality of uniform or non-uniform longitudinal ribs, a plurality of uniform or non-uniform ribs having a spiral configuration around a circumference of the inner surface, a plurality of ribs having a non-uniform pattern on the inner surface, a plurality of flat surfaces, a uniform or non-uniform roughened surface, a plurality of particulates or beads embedded in the sidewall, or any combinations thereof. According to certain embodiments, texturizing at least a portion of the inner surface of the preform may comprise texturizing at least a portion of an inner surface of an injection molded preform and blow-molding the injection molded preform to provide a rolling diaphragm, such as a rolling diaphragm syringe configured from injecting a medical fluid for a medical procedure. The rolling diaphragm may be made from a medical grade PET. In certain embodiments, texturizing may include texturizing at least a portion of at least one of the two portions of the inner surface during a blow-molding process.

According to the various embodiments, reducing the friction between the contacting portions of the rolled inner surface of the flexible sidewall of the rolling diaphragm may reduce or eliminate audible noise, such as an undesired audible squeaking noise, during a rolling and/or an unrolling of the rolling diaphragm, for example as the opposing contacting surfaces of the rolled inner surfaces slide over each other when moved relative to each other. According to certain embodiments, the coefficient of friction (μ) between contacting portions of the rolled inner surface of the flexible sidewall having the at least one surface texturization feature as the flexible sidewall is rolled or unrolled may range from 0.10 to 2.30. The value of the reduction of the coefficient of friction (μ) may depend on the type and polymeric structure of the medical grade plastic, for example the type or structure of a medical grade PET.

In certain embodiments, texturizing at least a portion of at least one of the two portions of the inner surface of the blow-molded rolling diaphragm syringe comprises texturizing the at least a portion of at least one of the two portions of the inner surface during a rolling process when a flexible sidewall of the blow-molded rolling diaphragm syringe rolls upon itself when acted upon by a piston such that an outer surface of the sidewall is rolled in a radially inward direction as the piston is advanced from a proximal end to a distal end of the blow-molded rolling diaphragm syringe

Still other embodiments of the present disclosure may be directed to methods for removing entrapped air between contacting portions of a rolled inner surface of a flexible sidewall of a rolling diaphragm. The method comprises texturizing at least a portion of the inner surface with at least one surface texturization feature selected from the group consisting of a plurality of uniform or non-uniform longitudinal ribs, a plurality of uniform or non-uniform ribs having a spiral configuration around a circumference of the inner surface, a plurality of ribs having a non-uniform pattern on the inner surface, a plurality of flat surfaces, a uniform or non-uniform roughened surface, a plurality of particulates or beads embedded in the sidewall, or any combinations thereof. The at least one surface texturization feature provides a fluid path for at least a portion of any entrapped air to escape from between the contacting portions of a rolled inner surface of the flexible sidewall of the rolling diaphragm. According to various embodiment, the at least one surface texturization feature may also reduce a coefficient of friction (μ) between contacting portions of the rolled inner surface of the flexible sidewall having the at least one surface texturization feature as the flexible sidewall is rolled or unrolled to a range from 0.10 to 2.30.

Still other embodiments of the present disclosure are directed to methods of forming rolling diaphragm syringes having at least one surface texturization feature, such as by blow-molding preforms having surface texturization features on at least a portion of a sidewall surface thereof. Various embodiments are directed toward molded preforms that are blow-molded to provide the rolling diaphragm syringes having at least one surface texturization feature on at least a portion of a sidewall thereof.

According to various embodiment, the present disclosure provides a rolling diaphragm syringe for receiving a medical fluid therein, the rolling diaphragm syringe comprising: a proximal end comprising a dome-shaped end wall configured to receive and engage a piston of a fluid injector, an annular portion extending proximally from a periphery of the end wall having a substantially cylindrical sidewall, and an outwardly flared portion extending radially and distally from the substantially cylindrical sidewall; an open distal end comprising a discharge neck; a flexible sidewall extending between the outwardly flared portion of the proximal end and the distal end along a longitudinal axis; and a piston engagement portion protruding proximally from a central portion of the end wall configured for engagement with the piston of the fluid injector, wherein the sidewall is flexible and rolls upon itself when acted upon by the piston such that an outer surface of the sidewall is rolled in a radially inward direction as the piston is advanced from the proximal end to the distal end, and wherein at least a portion of an inner surface of the flexible sidewall comprises at least one surface texturization feature.

In certain embodiments, the at least one surface texturization feature is selected from the group consisting of a plurality of uniform or non-uniform axial ribs, a plurality of uniform or non-uniform ribs having a spiral configuration around a circumference of the inner surface, a plurality of ribs having a non-uniform pattern on the inner surface, a uniform or non-uniform roughened surface, a plurality of particulates or beads embedded in the sidewall, or any combinations thereof.

Various aspects of the system and method for pressure calibration of the fluid injector are disclosed in one or more of the following numbered clauses:

Claus 1: A rolling diaphragm syringe for receiving a medical fluid therein, the rolling diaphragm syringe comprising: a closed proximal end wall for releasably engaging a piston of a fluid injector; a distal end having a neck and a fluid outlet; a flexible sidewall extending between the proximal end wall and the distal end, wherein the flexible sidewall rolls upon itself when acted upon by the piston such that the outer surface of the flexible sidewall rolls in a radially inward direction as the piston is advanced from the proximal end to the distal end and unrolls in a radially outward direction as the piston is retracted from the distal end to the proximal end; and at least one surface texturization feature on at least a portion of an inner surface of the flexible sidewall.

Clause 2: The rolling diaphragm syringe of clause 1, wherein the rolling diaphragm syringe is made from a medical grade polyethylene terephthalate (PET).

Claus 3: The rolling diaphragm syringe of clause 1 or 2, wherein the at least one surface texturization feature reduces the coefficient of friction (μ) between contacting portions of the inner surface of the flexible sidewall as it is rolled or unrolled.

Clause 4: The rolling diaphragm syringe of clause 2 or 3, wherein the coefficient of friction (μ) between contacting portions of the inner surface of the flexible sidewall having the at least one surface texturization feature as the flexible sidewall is rolled or unrolled ranges from 0.10 to 2.30.

Clause 5: The rolling diaphragm syringe of any of clauses 1 to 4, wherein the at least one surface texturization feature is selected from the group consisting of a plurality of uniform or non-uniform longitudinal ribs, a plurality of uniform or non-uniform ribs having a spiral configuration around a circumference of the inner surface, a plurality of ribs having a non-uniform pattern on the inner surface, a plurality of flat surfaces, a uniform or non-uniform roughened surface, a plurality of particulates or beads embedded in the sidewall, or any combinations thereof.

Clause 6: The rolling diaphragm syringe of any of clauses 1 to 4, wherein the at least one surface texturization feature is a plurality of longitudinal ribs.

Clause 7: The rolling diaphragm syringe of clause 6, wherein the plurality of longitudinal ribs are uniformly or non-uniformly arranged circumferentially around the inner surface.

Clause 8: The rolling diaphragm syringe of clause 6, wherein the plurality of longitudinal ribs extend only partially from the proximal end to the distal end.

Clause 9: The rolling diaphragm syringe of clause 6, wherein at least a portion of the plurality of longitudinal ribs extend for different lengths and at different regions along the longitudinal axis.

Clause 10: The rolling diaphragm syringe of clause 6, wherein the plurality of longitudinal ribs have different heights along a length of the longitudinal axis of the rib.

Clause 11: The rolling diaphragm syringe of any of clauses 1 to 4, wherein the at least one surface texturization feature is a plurality of uniform or non-uniform ribs having a spiral configuration around a circumference of the inner surface extending along at least a portion of the longitudinal axis.

Clause 12: The rolling diaphragm syringe of any of clauses 1 to 11, wherein the at least one surface texturization feature maintains a fluid pathway between substantially parallel inner surfaces of the at least partially rolled rolling diaphragm syringe to allow air to escape from between the parallel inner surfaces during a rolling or unrolling process.

Clause 13: A preform for blow-molding a rolling diaphragm syringe, the preform comprising: a closed proximal end portion having a piston engagement feature configured for allowing releasable engagement between the rolling diaphragm syringe and a piston of a fluid injector; a distal end having a fluid outlet; a sidewall having an inner surface and an outer surface; and at least one preform texturization feature on at least a portion of one of the inner surface and the outer surface of the sidewall, wherein the at least one preform texturization feature forms an at least one surface texturization feature on at least a portion of an inner surface of a sidewall of the rolling diaphragm syringe.

Clause 14: The preform of clause 13, wherein the at least one preform texturization feature comprises a plurality of preform longitudinal ribs on at least a portion of the inner surface of the sidewall, wherein the plurality of preform longitudinal ribs form a plurality of longitudinal ribs or a plurality of spiral ribs on at least a portion of the inner surface of the sidewall of the rolling diaphragm syringe.

Clause 15: The preform of clause 13, wherein the at least one preform texturization feature comprises a plurality of preform longitudinal ribs on at least a portion of the outer surface of the sidewall, wherein the plurality of preform longitudinal ribs form a plurality of longitudinal ribs or a plurality of spiral ribs on at least a portion of the outer surface of the sidewall of the rolling diaphragm syringe, and wherein the plurality of longitudinal ribs or the plurality of spiral ribs on at least a portion of the outer surface of the sidewall of the rolling diaphragm syringe are converted to a plurality of longitudinal ribs or a plurality of spiral ribs on at least a portion of the inner surface of the sidewall of the rolling diaphragm syringe during a blow-molding process.

Clause 16: The preform of any of clauses 13 to 15, wherein the at least one preform texturization feature on at least the portion of one or both of the inner surface and the outer surface of the sidewall are formed during an injection molding process using a mold having corresponding grooves on the core structure or the outer mold cavity.

Clause 17: The preform of any of clauses 13 to 15, the at least one preform texturization feature comprises a plurality of preform longitudinal ribs on at least a portion of the inner surface of the sidewall, wherein the plurality of preform longitudinal ribs are formed by etching a plurality of longitudinal grooves on the at least a portion of the inner surface of the sidewall during removing of an injection mold core structure having a corresponding plurality of groove etching members.

Clause 18: A method for reducing friction between contacting portions a rolled inner surface of a flexible sidewall of a rolling diaphragm, the method comprising: texturizing at least a portion of the inner surface of the flexible sidewall with at least one surface texturization feature selected from the group consisting of a plurality of uniform or non-uniform longitudinal ribs, a plurality of uniform or non-uniform ribs having a spiral configuration around a circumference of the inner surface, a plurality of ribs having a non-uniform pattern on the inner surface, a plurality of flat surfaces, a uniform or non-uniform roughened surface, a plurality of particulates or beads embedded in the sidewall, or any combinations thereof.

Clause 19: The method of clause 18, wherein texturizing at least a portion of the inner surface comprises: texturizing at least a portion of an inner surface of an injection molded preform; and blow-molding the injection molded preform to provide the rolling diaphragm.

Clause 20: The method of clause 18 or 19, wherein the rolling diaphragm is a rolling diaphragm syringe configured for injecting a medical fluid for a medical procedure.

Clause 21: The method of any of clauses 18 to 20, wherein the rolling diaphragm is made from a medical grade PET.

Clause 22: The method of any of clauses 18 to 21, wherein reducing the friction between the contacting portions of the rolled inner surface of the flexible sidewall of the rolling diaphragm reduces or eliminates an audible squeak during a rolling or an unrolling of the rolling diaphragm.

Clause 23: The method of any of clauses 18 to 22, wherein a coefficient of friction (μ) between contacting portions of the rolled inner surface of the flexible sidewall having the at least one surface texturization feature as the flexible sidewall is rolled or unrolled ranges from 0.10 to 2.30.

Clause 24: The method for removing entrapped air between a rolled inner surface of a flexible sidewall of a rolling diaphragm, the method comprising: texturizing at least a portion of the inner surface with at least one surface texturization feature selected from the group consisting of a plurality of uniform or non-uniform longitudinal ribs, a plurality of uniform or non-uniform ribs having a spiral configuration around a circumference of the inner surface, a plurality of ribs having a non-uniform pattern on the inner surface, a plurality of flat surfaces, a uniform or non-uniform roughened surface, a plurality of particulates or beads embedded in the sidewall, or any combinations thereof, wherein the at least one surface texturization feature provides a fluid path for the entrapped air to escape between the rolled inner surface of the flexible sidewall of the rolling diaphragm.

Clause 25: The method of clause 24, wherein the at least one surface texturization feature reduces a coefficient of friction (μ) between contacting portions of the rolled inner surface of the flexible sidewall having the at least one surface texturization feature as the flexible sidewall is rolled or unrolled to a range from 0.10 to 2.30.

Other embodiments of the present disclosure will become apparent to one of skill in the art reading the disclosure herein including the one or more Figures herewith.

### BRIEF DESCRIPTION OF THE DRAWINGS

The attached Drawings illustrate various features and non-limiting embodiments the rolling diaphragm syringes and preforms according to certain embodiments of the present disclosure.

FIGS. 1A to 1C illustrate an embodiment of a rolling diaphragm syringe in a completely unrolled configuration, including a perspective drawing (FIG. 1A), a cross sectional drawing (FIG. 1B) and a view along the longitudinal axis from the distal end of the rolling diaphragm syringe (FIG. 1C);

FIGS. 2A to 2C illustrate an embodiment of a rolling diaphragm syringe in a rolled configuration, including a perspective drawing (FIG. 2A), a cross sectional drawing (FIG. 2B) and a view along a longitudinal axis from the distal end (FIG. 2C);

FIG. 3 illustrates a cross sectional drawing of an embodiment of a rolling diaphragm syringe in a partially rolled state after an initial unrolling and purge cycle;

FIGS. 4A and 4B illustrate an embodiment of the rolling diaphragm syringe having a surface texturization feature including longitudinal ribs in an unrolled configuration (FIG. 4A) and a rolled configuration (FIG. 4B);

FIGS. 5A and 5B illustrate an embodiment of the rolling diaphragm syringe having a surface texturization feature including partial longitudinal ribs in an unrolled configuration (FIG. 5A) and a rolled configuration (FIG. 5B);

FIGS. 6A and 6B illustrate an embodiment of the rolling diaphragm syringe having a surface texturization feature including spiral ribs in an unrolled configuration (FIG. 6A) and a rolled configuration (FIG. 6B);

FIGS. 7A and 7B illustrate an embodiment of the rolling diaphragm syringe having a surface texturization feature including a dimpled texture in an unrolled configuration (FIG. 7A) and a rolled configuration (FIG. 7B);

FIGS. 8A and 8B illustrate an embodiment of an injection molded non-texturized preform for blow-molding a rolling diaphragm syringe including a perspective drawing (FIG. 8A) and a cross sectional drawing (FIG. 8B);

FIG. 9 illustrates an embodiment of a texturized preform having longitudinal ribs on an inner surface thereof;

FIG. 10 illustrates an embodiment of a texturized preform having longitudinal ribs on a proximal portion of an inner surface thereof;

FIG. 11 illustrates an embodiment of a texturized preform having longitudinal ribs of random lengths and position on an inner surface thereof;

FIG. 12 illustrates an embodiment of a texturized preform having spiral ribs on an inner surface thereof;

FIGS. 13A and 13B illustrates an embodiment of a texturized preform having longitudinal ribs on an outer surface thereof, wherein FIG. 13A shows a side view and FIG. 13B shows a terminal cross-sectional view from the distal end;

FIG. 14 illustrates an embodiment of a texturized preform having a texturizing material on an inner surface thereof;

FIG. 15 illustrates an injection mold core structure having longitudinal grooves for imparting at least one texturization feature onto a preform;

FIG. 16 illustrates an injection mold core structure having spiral grooves for imparting at least one texturization feature onto a preform;

FIG. 17 illustrates an injection mold core structure having groove cutting surfaces at a distal portion of the core for imparting at least one texturization feature onto a preform during core removal;

FIG. 18 illustrates an injection mold core structure having a polygonal cross-section for imparting at least one texturization feature onto a preform;

FIGS. 19A and 19B illustrate two embodiments an injection mold core structure having groove forming structures having a non-uniform depths for imparting at least one texturization feature onto a preform, wherein FIG. 19A structures form deeper grooves at a distal end and FIG. 19B structures form deeper grooves at a proximal end of the core;

FIGS. 20A and 20B illustrate two embodiments of stretch rods for stretch blow-molding a syringe according to various embodiments. FIG. 20A illustrates a stretch rod having a plurality of holes for particulate blasting an inner surface of the syringe and FIG. 20B illustrates a stretch rod having texturizing features thereon;

FIG. 21 illustrates a method for forming a plurality of spiral ribs on an inner surface during a stretch blow-molding process;

FIG. 22 illustrates a textured roll plunger for a rolling apparatus for imparting at least one texturization feature on an inner surface of a sidewall of a syringe;

FIGS. 23A and 23B illustrate a polymeric sidewall having particles of a different material in a preform (FIG. 23A) and the resulting textured surface of a blow-molded syringe (FIG. 23B);

FIG. 24 is a microscope cross-sectional image of a rolled flexible sidewall having no surface texturization features;

FIGS. 25A to 25D are microscope cross-sectional images of a distal rolled sidewall having a longitudinal ribbed surface texturization feature (FIG. 25A), a proximal rolled sidewall having a longitudinal ribbed surface texturization feature (FIG. 25B), a rolled distal and proximal sidewall where each sidewall include longitudinal ribbed surface texturization features (FIG. 25C), and rolled distal and proximal sidewall where only the proximal sidewall includes longitudinal ribbed surface texturization features (FIG. 25D); and

FIGS. 26A to 26C are graphs comparing coefficient of friction to squeak rating for various surface texturization features during partial fill (Phase 1, FIG. 26A), air purge (Phase 2, FIG. 26B), and full fill (Phase 3, FIG. 26C).

### DETAILED DESCRIPTION

The illustrations generally show preferred and non-limiting aspects of the systems and methods of the present disclosure. While the description presents various aspects of the devices, it should not be interpreted in any way as limiting the disclosure. Furthermore, modifications, concepts, and applications of the disclosure's aspects are to be interpreted by those skilled in the art as being encompassed, but not limited to, the illustrations and descriptions herein.

The following description is provided to enable those skilled in the art to make and use the described aspects contemplated for carrying out the disclosure. Various modifications, equivalents, variations, and alternatives, however, will remain readily apparent to those skilled in the art. Any and all such modifications, variations, equivalents, and alternatives are intended to fall within the spirit and scope of the present disclosure.

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures. When used in relation to a syringe, a pressure jacket, and/or a rolling diaphragm syringe, the term “proximal” refers to a portion of a syringe, a pressure jacket, and/or a rolling diaphragm syringe nearest to an injector when a syringe, a pressure jacket, and/or a rolling diaphragm syringe is oriented for connecting to the injector. The term “distal” refers to a portion of a syringe, a pressure jacket, or a rolling diaphragm syringe farthest away from the injector when the syringe, the pressure jacket, or the rolling diaphragm syringe is oriented for connecting to an injector. The term “radial” refers to a direction in a cross-sectional plane normal to a longitudinal axis of the syringe, the pressure jacket, or the rolling diaphragm syringe extending between proximal and distal ends. The term “circumferential” refers to a direction around an inner or outer surface of a sidewall of the syringe, the pressure jacket, or the rolling diaphragm syringe. The term “flexible”, when used in connection with a rolling diaphragm syringe, means that at least a portion of the rolling diaphragm syringe, such as a sidewall of the rolling diaphragm syringe, is capable of bending or being bent to change a direction in which it extends. The terms “roll over”, “rolling over”, and “rolls upon itself” refer to an ability of a portion of the rolling diaphragm syringe, such as a proximal end portion of the rolling diaphragm syringe, to bend approximately 180 relative to a second portion of the rolling diaphragm syringe, such as a distal portion of a sidewall of a rolling diaphragm syringe, when urged by a piston of a fluid injector. It is to be understood, however, that the disclosure may assume alternative variations and step sequences, except where expressly specified to the contrary. It is also to be understood that the specific devices and processes illustrated in the attached drawings, and described in the following specification, are simply exemplary aspects of the disclosure. Hence, specific dimensions and other physical characteristics related to the aspects (i.e., aspects, variants, variations) disclosed herein are not to be considered as limiting. As used herein, the term “substantially” means to within plus or minus 5% variation. For example, two surfaces denoted as “substantially parallel” can be up to plus or minus 5 degrees from parallel. As used herein, the term “rib” when references in relation to a surface texturization feature may also include a portion of an inner sidewall located between two adjacent radial grooves in the sidewall. For example, a rib may be formed by material projecting in a radially inward or radially outward direction relative to the sidewall or may be formed by the material of the sidewall that is located between two grooves that radially project into the material of the sidewall. As used herein, the process of blow-molding may include stretch blow-molding where a stretch rod in utilized during the blow-molding process.

Referring to the drawings in which like reference characters refer to like parts throughout the several views thereof, the present disclosure is generally directed to a syringe configured as a rolling diaphragm syringe 10 configured to be connected to a syringe rolling apparatus or a fluid injector. For example, the syringe 10 may be configured to be received within a pressure jacket mounted to the apparatus or injector for providing radial and axial support for the syringe 10 during rolling or during an injection. Examples of rolling diaphragm syringes suitable for incorporation of at least one texturization feature according to the present disclosure are described in PCT International Publication Nos. WO 2015/164783; WO 2016/172467; and WO 20018/075386, and in U.S. Provisional Application Ser. No. 62/632,026, the disclosures of each of which are incorporated herein in their entireties by this reference.

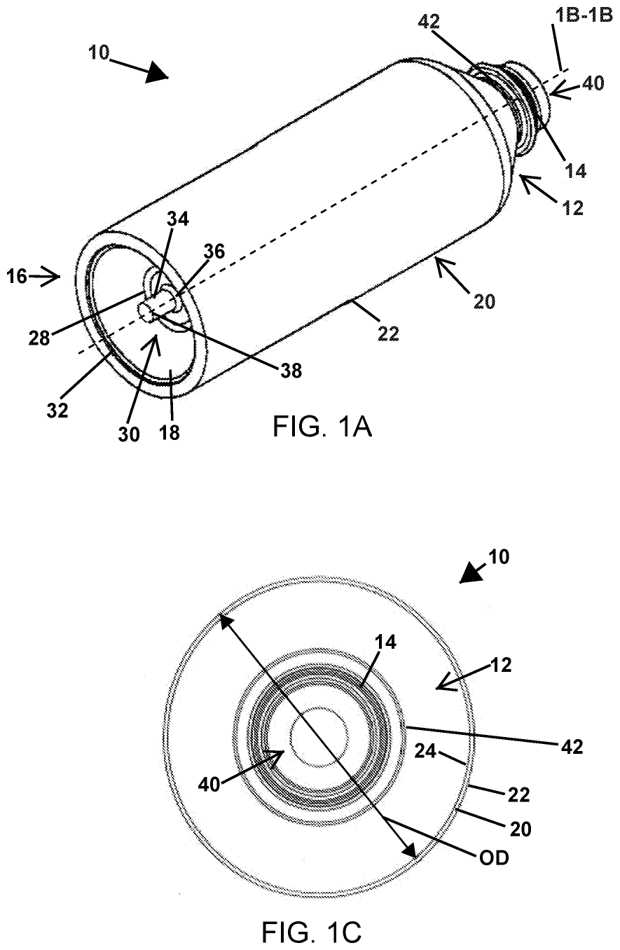

With reference to FIGS. 1A-3, the rolling diaphragm syringe 10 generally includes a hollow body defining an interior that includes a forward or distal end 12 including an discharge neck 14 having a fluid outlet 40, a rearward or proximal end 16 having a closed end wall 18, and a flexible sidewall 20 having an outer surface 22 and an inner surface 24 extending therebetween. The syringe 10 can be any suitable length L, which can be determined either by the length of the sidewall 20 or by the extent that the rolling diaphragm has been rolled and can have any interior volume depending on the fluid volume being injected and size of fluid injector being used. The syringe 10 may include a retention flange 42 located on the discharge neck 14 to assist in retaining the syringe 10 in the injector.

In some examples, the outer diameter OD may be dimensioned such that the rolling diaphragm syringe 10 fits within an interior space defined by the throughbore and an inner surface of a pressure jacket (not shown) of a fluid injector. In one aspect, the rolling diaphragm syringe 10 may fit snuggly within the pressure jacket such that the outer surface 22 of the rolling diaphragm syringe 10 abuts the inner surface of the walls of the pressure jacket. In another aspect, the rolling diaphragm syringe 10 fits loosely within the pressure jacket such that there is a gap between at least a portion of the outer surface 22 of the rolling diaphragm syringe 10 and the inner surface of the pressure jacket. The rolling diaphragm syringe 10 may be expanded under pressure such that the outer surface 22 of the rolling diaphragm syringe 10 abuts or contacts the inner surface of the pressure jacket.

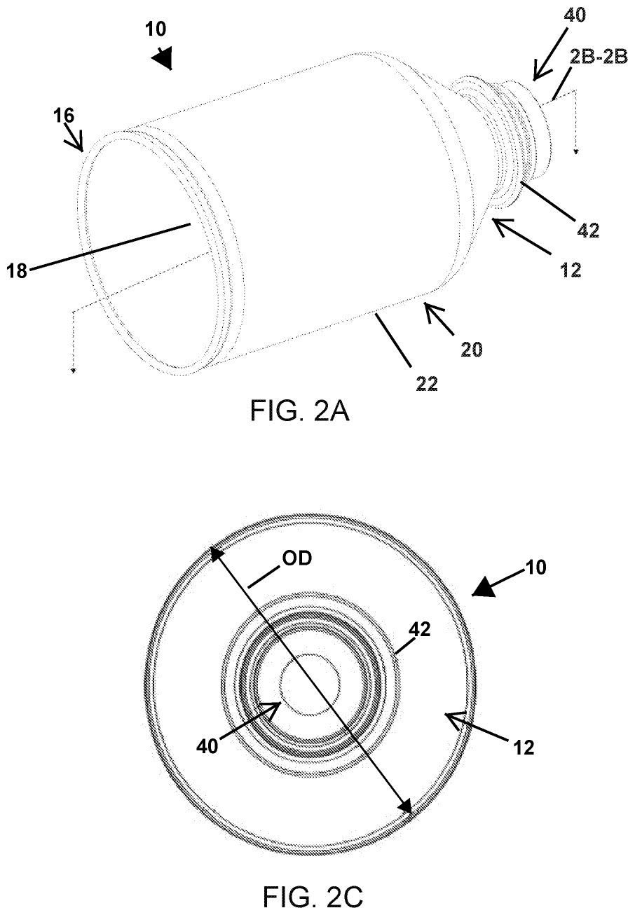

Referring to FIG. 3, a cross-sectional illustration of an embodiment of the rolling diaphragm syringe 10 in a partially unrolled or expanded state after initial rolling. In FIG. 3, the proximal end 16 of the flexible sidewall 20 begins to roll in an inward direction as a piston in contact with the proximal end wall 18 moves in the distal direction. As the flexible sidewall 20 begins to roll, a circumferential bead 48 where the flexible wall 20 rolls is formed. The proximal end 16 and the end wall 18 are slightly recessed within the syringe sidewall 20, such that a proximal-most portion of the syringe 10 flares outwardly beyond the sidewall 20. In that case, the cylindrical portion 46 is adjacent to the sidewall 20. A flared diameter OD3 of the proximal-most portion of the syringe 10 may be slightly larger than the syringe diameter OD.

The rolling diaphragm syringe 10 may be made of any suitable medical-grade plastic or polymeric material, desirably a clear or substantially translucent plastic material, such as, but not limited to, polypropylene random copolymer, polypropylene impact copolymer, polypropylene homopolymer, polypropylene, polyethylene terephthalate (PET), POM, ABS, HPDE, nylon, cyclic olefin copolymer, multilayer polypropylene, polycarbonate, ethylene vinyl acetate, polyethylene, and the like. The material of the rolling diaphragm syringe 10 is desirably selected to meet the required tensile and planar stress requirements, water vapor transmission, and chemical/biological compatibility.

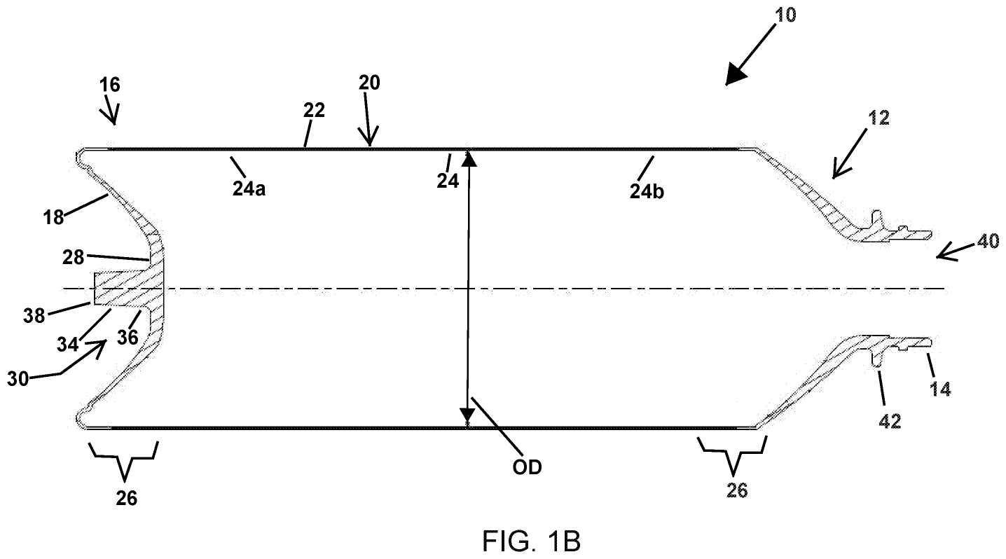

According to specific embodiments, the syringe may be polyethylene terephthalate (PET) or other medical graph polymer, such as a medical grade polymer selected from EASTAR™ MN021 and EASTAR™ MN052, commercially available from Eastman Chemical Co. One feature of PET and other polymeric materials is that when two smooth surfaces of PET or other polymeric material slide past and over each other, the coefficient of friction (μ) may be significant and can result in an increased force requirement to overcome the static friction between the two surfaces so that they can slide easily relative to one another. An additional feature may be that the two surfaces emit audible noise, such as a squeak or other high pitched noise, as the surfaces slide relative to each other due to kinetic friction between the surfaces. This issue cannot be solved by simply increasing the distance between the two surfaces, since when the distance between the two surfaces becomes too large, the flexible sidewalls may pleat or form creases during rolling and unrolling. For unrolling, this issue may become more prevalent since a partial vacuum if formed in the syringe interior which causes the sidewall to bow inwardly, further increasing the likelihood of pleating. For various embodiments of the rolling diaphragm syringe 10, two portions of the inner surface 24 of the syringe 10 must roll and slide over each other while in abutting contact as the syringe is rolled and unrolled, for example during an injection procedure. For example, as illustrated in FIGS. 1B and 2B, in the unrolled and rolled configuration of the rolling diaphragm syringe 10, respectively, proximal inner surface 24a is rolled and slides over distal inner surface 24b during the rolling and unrolling process. Frictional contact as the two inner surfaces, such as 24a, 24b, slide over each other results in requiring more force to roll and unroll the syringe and may result in undesired audible noise, such as a loud squeaking during an injection procedure when the frictional forces are present. In the present disclosure, the surface friction and squeaking may be minimized by texturizing, with at least one surface texturization feature, at least a portion of the inner surface(s) 24 of the syringe that contact and slide relative to another portion of the inner surface. Further, other undesired phenomena which may occur during rolling, such as entrapment of air between at least portion of the proximal inner surface 24a and the distal inner surface 24b when rolled, may be minimized by texturizing at least a portion of the inner surface(s) 24 of the syringe with at least one surface texturization feature, such that a fluid path is established to allow the entrapped air to escape from between the contacting portions of the inner surface 24. Without intending to be limited by any particular mechanism, it is believed that texturization of at least a portion of the inner surfaces 24 of the rolling diaphragm syringe 10 with at least one surface texturization feature may reduce surface-to-surface contact area, thereby reducing the coefficient of friction (μ) as the surfaces slide relative to each other. As the coefficient of friction (μ) is reduced, undesired squeaking or other audible noise may be minimized or eliminated.

In some examples, the rolling diaphragm syringe 10 may be reusable, meaning that the syringe 10 can be rolled and unrolled multiple times before being disposed of or recycled. For example, the rolling diaphragm syringe 10 can be filled as described above, rolled to deliver fluid contained therein to the patient, and then unrolled and re-filled several times to deliver additional doses of fluid to the patient. Alternatively, when utilized with a single-patient fluid path with appropriate check valves and connectors to prevent cross-contamination between patients, the rolling diaphragm syringe 10 may be used as a multi-patient syringe, with the single-patient portion of the fluid path being disposed of in between injection procedures. Alternatively, the rolling diaphragm syringe 10 may be a single-use component that is disposed of after each patient use.

According to various embodiments of the present disclosure, the rolling diaphragm syringe 10 described herein may comprise at least one surface texturization feature on at least a portion of an inner surface 24 of the flexible sidewall 20 of the rolling diaphragm syringe 10. The at least one surface texturization feature is selected from the group consisting of a plurality of uniform or non-uniform longitudinal ribs, a plurality of uniform or non-uniform ribs having a spiral configuration around a circumference of the inner surface, a plurality of ribs having a non-uniform pattern on the inner surface, a plurality of flat surfaces, a uniform or non-uniform roughened surface, a plurality of particulates or beads embedded in the sidewall, or any combinations thereof. As used herein the term “uniform” means that each surface texturization feature has substantially the same shape over the entirety of the surface texturization feature, for example, a uniform longitudinal rib will have substantially the same height, length, and/or width. As used herein the term “non-uniform” means that at least one feature of a surface texturization feature has a different shape or size at at least a portion of the texturization feature, for example, a non-uniform longitudinal rib may differ in height, width, and/or length at at least a portion of the rib. Non-limiting examples of non-uniform longitudinal ribs include, for example, ribs extending along only a portion of the syringe sidewall, ribs that have different heights (e.g., extend a different radial distance into the interior volume of the syringe) along the length of the rib, ribs that have different widths (e.g., are wider at one portion than at another) along the length of the rib, ribs that may have gaps along the length of the rib, etc. Non-uniformity of the surface texturization feature may provide a benefit, such as allowing the preform or syringe to be more readily removed from the injection or blow mold, respectively by providing a suitable draft angle for mold removal and/or preventing/limiting undercuts in the molding process.

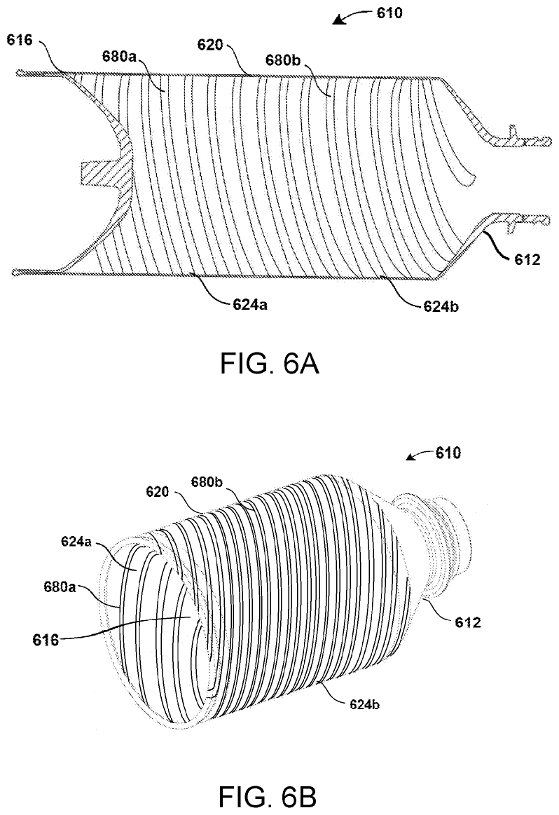

In some examples, the rolling diagraph syringe 10 is formed from a preform 800 (see e.g., FIG. 8A) by a blow-molding process, such as a stretch blow-molding process, in which a preform 800, such as a preform produced by injection molding, is elongated and enlarged by a combination of heating and stretching using a metal core and radial expansion by air pressure until the blow-molded plastic contacts the surface of the mold to shape the syringe. Embodiments of a rolling diaphragm syringe 10 formed by stretch blow-molding prior to initial rolling is shown in FIGS. 1A-1C. An exemplary stretch blow-molding process is described in International Patent Application No. WO 2015/066506 entitled “Blow-Molded Syringe for Use with An Injector”, the disclosure of which is incorporated herein by reference in its entirety. Alternative methods for forming rolling diaphragm syringes, which may also be used with the methods for initial rolling disclosed herein, are disclosed in International Publication No. WO 2016/172467 entitled “Syringe with Rolling Diaphragm”.

According to certain embodiments, described in detail herein, the inner surface of the preform 800 may be texturized prior to the blow-molding process. For example, at least one preform texturization feature may be included onto at least a portion of an inner surface 824 and/or an outer surface 822 of the preform 800 during an injection molding process (see FIG. 8B). The resulting preform having the at least one texturization feature may then be blow-molded to form a rolling diaphragm syringe and the resulting inner surface of the blow-molded syringe may have a desired at least one texturization feature. Alternatively, or in addition, in other embodiments, the inner surface 24 of the syringe may be texturized with at least one texturization feature during the blow-molding process. In still other embodiments, the inner surface 24 of the blow-molded syringe may be texturized with at least one texturization feature after the blow-molding process, for example, by texturizing the inner surface 24 prior to initial rolling of the syringe 10 or by texturizing the inner surface 24 during a rolling process.

According to certain embodiments, following a stretch blow-molding process, the syringe 10 can be structurally modified by the initial rolling action in which, as described herein, an unfilled syringe (e.g., a syringe 10 that does not contain a medical solution) is contacted by a piston and rolled to mold or reform certain structural features. A syringe 10, after initial rolling in a contracted or rolled state, is shown in FIGS. 2A to 2C. A syringe 10, after initial rolling in its expanded or unrolled state, is shown in FIG. 3. As described herein, during the initial rolling action according to certain embodiments, at least one surface texturization feature may be imparted to at least a portion of an inner surface 24 of the flexible sidewall 20 of the syringe 10.

Following the initial rolling, the syringe 10 having the at least one texturization feature may be stored and/or shipped to customers in the compressed or rolled state. Providing the rolling diaphragm syringe 10 in the compressed or rolled configuration provides economic benefits by reducing space required for storing, transporting, and disposal of manufactured syringes. Costs are reduced since the rolling diaphragm syringes 10 disclosed herein use less raw materials for manufacture and weigh less than similarly sized conventional disposable syringes known in the art and used with fluid injectors. When ready for use in a fluid injector, the syringe 10 is inserted into a pressure jacket and engaged with a piston of a fluid injector. The syringe 10 is at least unrolled by proximal retraction of a piston of the injector to draw a desired volume of fluid into the syringe interior. The fluid-filled unrolled syringe 10, shown in FIG. 3, may then deliver the fluid to a patient but distal movement of the piston and rolling of the flexible sidewall 20 by an amount necessary to deliver the prescribed volume of fluid. After the injection, the rolled syringe 10 may be disposed of or may be reused for a limited time in a multi-patient delivery scenario by repeating the filling process.

With continued reference to FIGS. 1A-3, the flexible sidewall 20 of the syringe 10 is an elongated substantially cylindrical structure, which defines a soft, pliable or flexible, yet self-supporting body that is configured to roll upon itself, as a “rolling diaphragm”, under a force, such as the action of a piston of a fluid injector and/or syringe rolling apparatus, for example a piston releasably attached to or abutting the end wall 18 of the syringe 10. In particular, the sidewall 20 of the rolling diaphragm syringe 10 is configured to roll such that its outer surface 22 is rolled and inverted in a radially inward direction as the piston is moved in a distal direction, such that a rolled portion 22a (shown in FIG. 2B) of the outer surface 22 and rolled portion 24a of the inner surface 24 are brought near to an unrolled portion 24b and 22b, respectively (shown in FIG. 2B), of the inner surface 24 and outer surface 22. The outer surface 22 unrolls and unfolds in the opposite manner in a radially outward direction as the end wall 18 is retracted or moves in a proximal direction. According to various embodiments of the present disclosure, at least a portion of the inner surface 24 of the sidewall 20 and optionally the end wall 18 and/or the distal conical portion of 12 may have at least one texturization feature to form a texturized surface, or a combination of a smooth surface and a textured surface. During rolling and unrolling, the textured surface reduces a surface area of contact between the rolled portion 24a and unrolled portion 24b of the inner surface 24. When contact occurs between portions of the inner surface 24, the at least one texturization feature on the inner surface 24 reduces a coefficient of friction (μ), and thereby any frictional forces between the portions 24a, 24b of the inner surface 24. Due to properties of the syringe polymeric material, friction between contacting portion of the inner sidewall 24 can reduce rolling efficiency of the syringe 10, place additional strain on an injector motor, and cause undesired audible noise (e.g., squeaks or squeals) when the portions 24a, 24b move and slide in contact relative to one another. In certain embodiments, the rolling diaphragm syringe 10 having a plurality of surface texturization features to reduce the friction between contacting portion of the proximal and distal inner surfaces 24a, 24b of the sidewall 22 may also result in less particulate generation as the inner surfaces 24 of the sidewalls 22 more readily slide over one another during rolling and unrolling processes.

In some examples, the proximal and distal ends 16, 12 of the syringe 10 may be thicker than the flexible sidewall 20 of the syringe 10 to accommodate forces associated with loading and delivering a fluid under pressure. According to these embodiments, transition zones 26 between the thicker distal or proximal ends and the thinner sidewall 20 may be short in length relative to the sidewall 20, which may be accomplished during the stretch blow-molding process. For example, as shown in FIG. 1B, the cylindrical portion of the sidewall 20 can taper towards the proximal and distal ends 16, 12 of the syringe 10, such that middle cylindrical portions of the sidewall 20 may be from about 150% to 350% thinner than the portions of the sidewall 20 adjacent to the proximal and distal ends 16, 12 of the syringe 10. In other examples, the sidewall 20 has a substantially smooth outer surface 22 and a constant thickness along a majority of its longitudinal length. However, applicants have found that a smooth inner surface 24 may result in undesired frictional forces and entrapped air during a rolling/unrolling process, as described herein. According to various embodiments herein, the inner surface 24 of the sidewall 20 can include at least one surface texturization feature, such as one or more ribs, including a plurality of ribs, provided thereon to facilitate rolling (i.e., by reducing a coefficient of friction (μ) of the texturize surface) and/or to maintain spacing between rolled and unrolled portions of the sidewall 20. According to certain embodiments, the at least one surface texturization feature, such as a plurality of ribs, may result in reduction of air that may become entrapped between the proximal inner surface 24a and the distal inner surface 24b when the syringe in filled. For example, in certain aspects, the at least one surface texturization feature, such as a plurality of ribs, may provide one or more fluid pathways for any entrapped air to escape the regions between the rolled proximal inner surface 24a and the distal inner surface 24b of the syringe when the injector head and attached rolling diaphragm syringe(s) 10 is placed in an upright position (i.e., with the distal portion 12 of the syringe 10 pointing upwards). Entrapped air may then travel upwards towards the distal fluid outlet 40 where it may be purged during a priming/purging operation that removes air from the injection system.

In some examples, the closed end wall 18 may have a concave, dome-shaped structure to facilitate initiation of the inversion or rolling of the sidewall 20 and/or to provide a receiving space or pocket to receive a distal end of a piston. Further, the end wall 18 may have a non-uniform thickness, for example in a radial direction extending from a central longitudinal axis of the rolling diaphragm syringe 10. For example, at least a portion of the end wall 18 may be thicker near central portion 28 and thinner near the connection with the sidewall 20. The closed end wall 18 may be shaped to interface directly with a piston of a fluid injector. In particular aspects, the piston may be shaped to substantially match the shape of the closed end wall 18 or, alternatively and as described in detail herein, pressure from the piston as it is moved distally may conform the end wall 18 and/or other portions of the proximal end 16 of the syringe 10 to substantially match a shape of the piston, for example during the initiation of the rolling process.

In various embodiments, the end wall 18 may have a central portion 28 including a piston engagement portion 30 extending proximally therefrom, such as at an approximate midpoint of the central portion 28. In some aspects, a distal most end of the central portion 28 may be substantially flat over a partial radius of the closed end wall 18.

The piston engagement portion 30 may be configured for engagement with an engagement mechanism on a piston of the fluid injector (see, e.g., the disclosure of PCT Published Application Nos. WO 2015/164783; WO 2016/172467; and WO 20018/075386). In some aspects, the piston engagement portion 30 may include a stem 34 having a first end 36 connected at the central portion 28 and a second end 38 extending proximally from the first end 36. In certain embodiments, the stem 34 may include a protrusion configured for interacting with one or more engagement pins or surfaces of an engagement mechanism of a fluid injector that moves radially inward and outward to engage and disengage the stem 34 of the rolling diaphragm syringe 10.

With continued reference to FIGS. 1A-3, in some examples, the discharge neck 14 at the open distal end 12 of the syringe 10 is adapted to be received in an interior portion of a pressure jacket such that the discharge neck 14 is aligned with an outlet port of the pressure jacket. In that case, the discharge neck 14 may have a frusto-conical shape that gradually narrows from the cylindrical sidewall 20 to the discharge neck 14. In certain aspects, the discharge neck 14 may terminate in a fluid outlet 40.

A fluid injection or fluid delivery system includes a fluid injector, such as an automated or powered fluid injector, adapted to interface with one or more rolling diaphragm syringes 10 and pressure jackets with each syringe 10. Each of the syringes 10 may be independently filled with a medical fluid, such as an imaging contrast media, saline solution, or any desired medical fluid. The injector may be used during a medical procedure to inject the medical fluid (e.g., contrast media or saline solution) into the body of a patient by driving a piston into the at least one rolling diaphragm syringe 10 to cause the syringe 10 to roll in a distal direction to expel fluid therefrom. The piston can be sized to engage the end wall 18 of the syringe 10. The injector may be a multi-syringe injector, wherein two or more rolling diaphragm syringes 10 with corresponding pressure jackets are oriented in a side-by-side or other relationship and include corresponding pistons actuated by a linear actuator of the injector. In aspects with two rolling diaphragm syringes 10 and pressure jackets arranged in a side-by-side relationship and filled with two different medical fluids, the injector may be configured to deliver fluid from one or both of the rolling diaphragm syringes 10. In certain embodiments, the fluid injector may be configured to interface with either rolling diaphragm syringes, conventional plunger-containing syringes, or a combination of one or more rolling diaphragm syringes and one or more conventional plunger-containing syringes.

The injector may be enclosed within a housing formed from a suitable structural material, such as plastic or metal. The housing may be of various shapes and sizes depending on the desired application. For example, the injector may be a free-standing structure configured to be placed on the floor or may be a smaller design for placement on a suitable table or support frame. At least one fluid path set may be fluidly connected with a discharge neck 14 of the syringe(s) 10 for delivering medical fluid from the at least one rolling diaphragm syringe 10 through tubing to a catheter, needle, or other fluid delivery connection (not shown) inserted into a patient at a vascular access site. The tubing may be connected to the discharge neck by a unitary or removable cap, such as a cap described in PCT International Application No. PCT/US2018/050640, the disclosure of which is incorporated herein by this reference. Fluid flow into and from the syringe(s) 10 may be regulated by a fluid control module (not shown). The fluid control module may operate various pistons, valves, and/or flow regulating structures to regulate the delivery of the medical fluid, such as saline solution and contrast, to the patient based on user selected injection parameters, such as injection flow rate, duration, total injection volume, and/or ratio of contrast media and saline.

One example of a suitable front-loading fluid injector that may be used or modified for use with rolling diaphragm syringes 10 is disclosed in U.S. Pat. No. 5,383,858 which is incorporated by reference in its entirety. Other examples of relevant multi-fluid delivery systems that may be used or modified for use with the present system are found in U.S. Pat. Nos. 7,553,294 and 7,666,169; International Patent Application Publication Nos. WO 2012/155035 and 2015/164783; and United States Patent Application Publication No. 2014/0027009, the disclosures of which are incorporated herein by reference.

In use, syringe(s) 10 may be provided to a medical facility in the compressed or rolled state as shown in FIGS. 2A-2C. For example, the syringe(s) 10 may be initially rolled, prior to being shipped to the medical facility. During use, the rolled syringe(s) 10 may be placed in a pressure jacket(s) associated with the fluid injector and placed in fluid communication with a fluid source through a connector means, such as a cap, attached to the discharge neck 14 of the syringe 10. Once fluid communication with the fluid source is established, the injector may be actuated causing the piston to advance through the pressure jacket and engage the end wall 18 of the rolled syringe 10, such as by engaging the piston engagement member 30. Once the piston is engaged with the end wall 18, the piston may be retracted proximally through the pressure jacket causing the syringe 10 to unroll and draw fluid from the source into an interior of the syringe 10. Once the syringe 10 is filled with a desired volume of fluid, fluid delivery occurs by reversing the piston direction and moving in the distal direction. Specifically, the piston is advanced in the distal direction causing the syringe 10 to re-roll to the compressed or rolled state while expelling fluid from the discharge neck 14 of the syringe 10 for fluid delivery to the patient. As discussed herein, in some examples, the syringe(s) 10 are single-use syringes which are disposed of after each use. In other examples, the syringe(s) 10 can be reusable. In that case, the syringe(s) 10 can be re-filled by again retracting the piston in the proximal direction, to re-unroll the syringe 10, thereby drawing another dose of fluid from a fluid source into the syringe 10 for performing another fluid injection. As discussed previously, fluid is expelled from the syringe 10 by advancing the piston in the distal direction to roll the syringe 10. This process may be repeated multiple times as determined by best practices of the syringe manufacturer and/or the medical facility.

According to certain aspects, one focus of the present disclosure is to reduce or remove an undesired audible noise (e.g., a squeak) associated with rolling/unrolling of the rolling diaphragm syringe 10 upon itself. One solution according to various embodiments herein is to add at least one surface texturization feature to at least a portion of an inner surface 24 of the rolling diaphragm syringe 10. It has been observed that when unrolling the syringe 10 a slight vacuum is created within the syringe when fluid or air is drawn in causing the distal sides of the flexible sidewall 20, including the unrolled portion of the inner surface 24b to draw inwardly, thereby contacting and interfering with the rolled portion of the inner surface 24b as it is retracting. This results in surface area contact between the two portions of the inner surfaces 24a,24b that increases the frictional contact between the two portions of the inner surface 24a,24b as they slide over each other, requiring additional force to continue the unrolling/rolling process and has the additional undesired effect of causing audible noise in the form of a squeak as the plastic material, such as PET, rolls and the smooth PET inner surfaces 24a,24b slide relative to each other. This can also happen when there is a slight misalignment in the piston or the pressure jacket and even during the initial rolling process. The syringe may also squeak from the technician handling the syringe, for example as they are installing the syringe into the injector and causing the inner surfaces 24a,24b to contact. While this has no actual impact on the operation of the syringe, the technician may become concerned by the noise. One solution according to various embodiments herein is to impart some type of surface texture to the inner surface 24 of the syringe 10 to break up the surface area of contact between the smooth inner surfaces, resulting from the blow-molding process, which must roll across one another during the rolling or unrolling process. By adding at least one surface texturization feature to at least a portion of the inner surface 24, the contact surface area is reduced which reduces the coefficient of friction (μ) between the two surfaces 24a,24b and the energy to create the squeak. According to other embodiments, the friction force between the inner surfaces 24a,24b of the rolling diaphragm syringe 10 may be reduced, for example, by addition of a lubricant like a medical grade silicone to the inner surface 24, addition of a non-silicone lubricous coating such as for example TRIBOGLIDE to the inner surface 24, addition of a gas or liquid to physically or chemically etch or modify the inner surface of the syringe, or ensure the walls never touch through accurate alignment of the rolled surfaces, the pressure jacket and the injector piston.

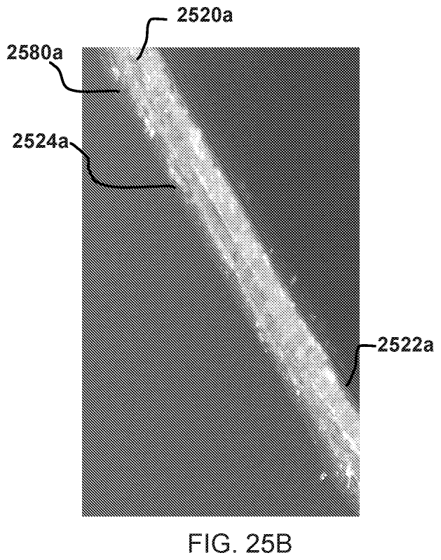

According to various embodiments of the present disclosure, adding at least one texturization feature to at least a portion of the inside surface finish may be accomplished in multiple ways (including combinations of different ways). According to certain embodiments, texture may be added to a core portion of the injection mold during the molding (injection molding) of the preform 800 and the resulting texture may be transferred to the inner surface 824 of the preform 800 (see, FIG. 8B), which upon blow-molding will transfer, potentially in significantly lesser extent (i.e., less depth) as the plastic is stretched during blow-molding process, onto at least a portion of the inner surface 24 of the blow-molded rolling diaphragm syringe 10. According to certain embodiments, addition of at least one texturization feature to the syringe 10 may be affected by imparting a heavy texture on the inside surface of the blow-mold which would transfer to the inner surface of the syringe through the outer surface contacting the textured blow-mold surface. According to other embodiments, addition of at least one texturization feature to the syringe 10 may be affected using a heavy texturized rolling plunger 2200 (see FIG. 22) during a rolling process, wherein at least one texturization feature 2210 is transferred from the rolling plunger 2200 through the polymer surface to at least a portion of the inner surface 24 of the syringe 10 (since the rolling plunger 2200 only contacts the proximal end of the syringe 10, the at least one texturization feature would transfer to the proximal inner surface 24a of the syringe 10. According to other embodiments, addition of at least one texturization feature to the inner surface of the pressure jacket of a rolling apparatus during an initial rolling process may also transfer at least one texturization feature to the inner surface 24 (through the polymer sidewall 20) as the syringe 10 is rolled and pressed against the inner surface of the pressure jacket. According to other embodiments, addition of at least one texturization feature to a finished syringe 10 may be affected by impacting the inner surface 24 with particulates, for example, by sandblasting, dry ice-blasting, ice-blasting, bead-blasting, and/or blasting with solid particulates of a contrast material or sodium chloride or other component of the medical fluid, a portion of the inner surface 24 of the syringe 10, or by chemically or physically (e.g., using an electromagnetic radiation source (E-beam, laser, plasma irradiation, etc.)) etching the polymeric material of the inner surface 24 of the syringe 10 to provide an appropriate texturization feature. Alternatively in other embodiments, the syringe 10 may be texturized to have at least one texturization feature by molding a “blotchy” syringe, for example, by using uneven heats at regions of the sidewall during the preform molding and/or the blow-molding process(es) to form regions of the syringe 10 having different chain structure or orientation resulting in texturized regions at various portions of the inner surface 24. In still another embodiment, a finished syringe may be re-heated and placed in a “texturing” mold to impart the at least one texturization feature to at least a portion of the inner surface 24 of the syringe 10.

Various plastic materials, for example medical-grade plastics such as PET, may have a high coefficient of friction (μ) when two surfaces of the material slide across each other, for example during a rolling or unrolling process of a rolling diaphragm syringe made therefrom. For example, EASTAR™ MN021 medical grade PET may have a coefficient of friction of μ=2.07 and EASTAR™ MN052 medical grade PET may have a coefficient of friction of μ=0.81. According to various embodiments, the coefficient of friction (μ) for the inner surfaces 24 having at least one texturization feature, for example inner PET surfaces, may be reduced by 40% to 90%, or in other embodiments from 50% to 80%. The amount of reduction of the coefficient of friction may be dependent on the grade or structure of the polymeric material (e.g., PET), for example, the coefficient of friction of MN021 may be reduced by from 50% to 90% and the coefficient of friction for MN052 may be reduced by from 20% to 60%. According to various embodiments, addition of at least one texturization feature to at least a portion of an inner surface 24 of the flexible sidewall 20 of the rolling diaphragm syringe 10 may result in a coefficient of friction (μ) that ranges from 0.10 to 2.30 and in certain embodiments from 0.10 to 1.20 or even from 0.30 to 1.20. According to embodiments herein, this can be accomplished by adding at least one texturization feature to at least a portion of an inner surface 24 of the flexible sidewall 20 of the rolling diaphragm syringe 10 during at least one of a preform injection molding process to form the preform 800, a stretch blow-molding process of the preform 800 to form the syringe 10, a rolling process of the blow-molded rolling diaphragm syringe 10 or during two or more of these processes. The two inner surfaces 24a, 24b of the flexible sidewall 20 slide against each other when rolling and unrolling of the syringe 10, creating an undesired audible noise as they frictionally contact each other. In certain embodiments, when the coefficient of friction between the two inner surfaces 24a, 24b of the flexible sidewall 20 is reduced to less than or equal to 1.00, no audible squeak is heard by the user. While both rolling and unrolling the syringe creates noise (squeaks), more noise may be experienced during the unrolling process because there is usually a partial vacuum created as the end wall 30 is retracted, which causes the distal portion of the flexible sidewall to drawn in and create even more interference/contact between the two inner walls 24a, 24b.

One approach according to various embodiments includes adding at least one texturization feature to at least a portion of the inner surface 24 of the sidewall 20 of a blow-molded syringe 10. According to the process (refer to FIGS. 8A and 8B), the preform 800 is first stretched (which will reduce the thickness of the plastic in the wall 820 of the preform 800) and blow-molded (which will further reduce the thickness of the plastic in the wall of the preform 800) to form the 3-dimensional structure of the rolling diaphragm syringe 10. One approach to provide the at least one texturization feature on the inner surface 24 may include creating a deep texture in the inner surface of the preform walls. However, this may require a large amount of draft (angle of the core) to be able to remove the core.

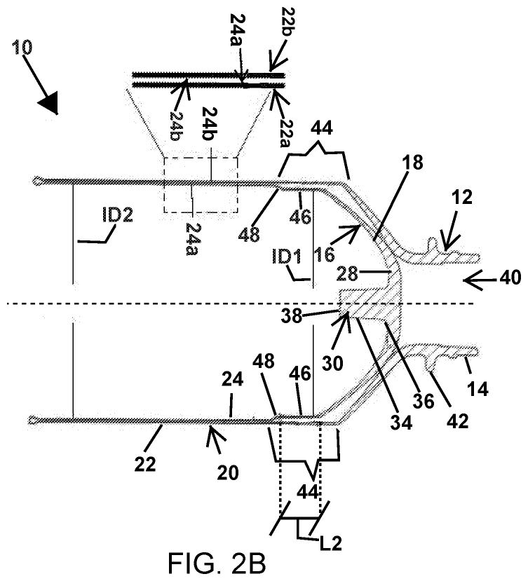

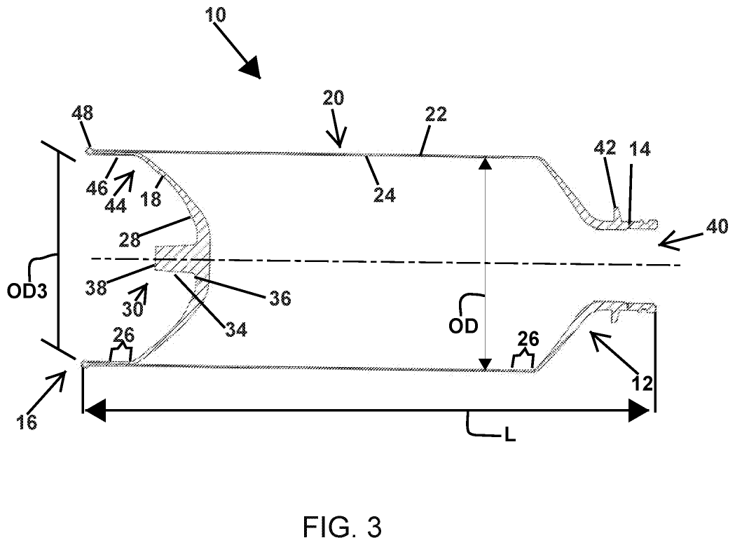

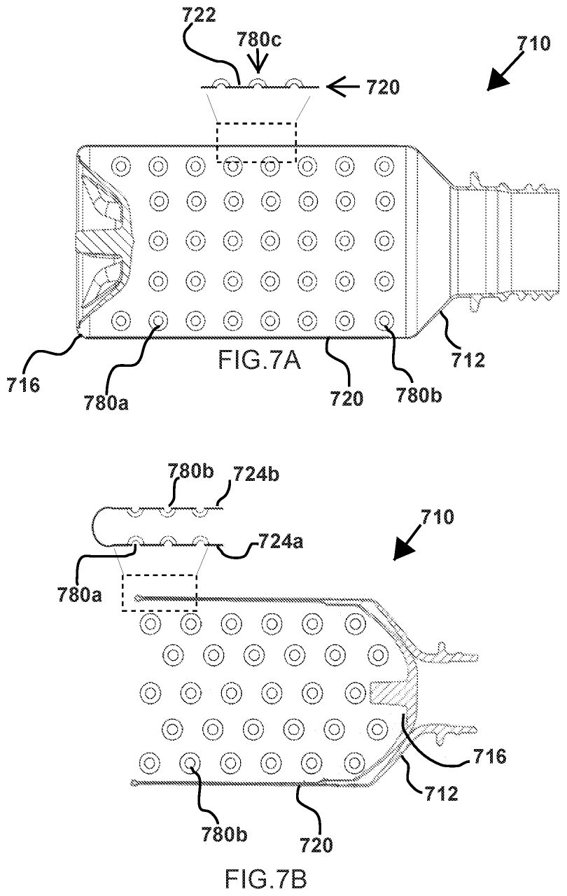

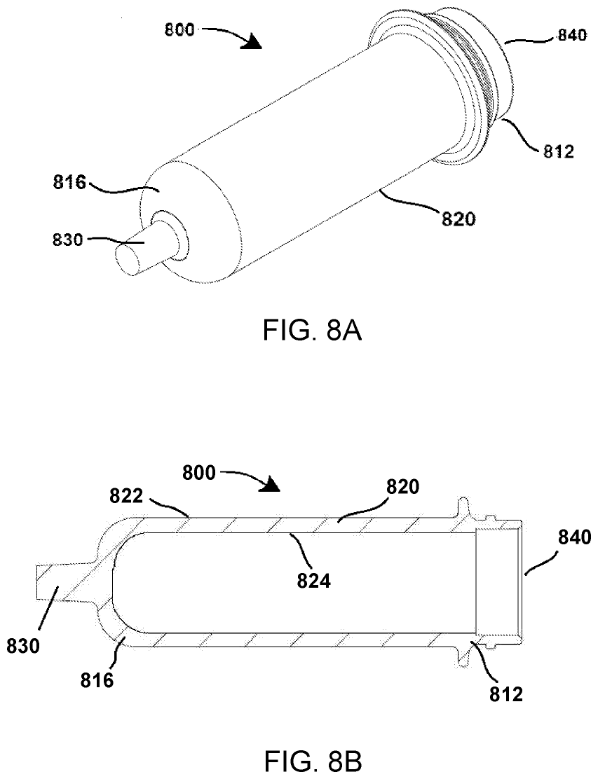

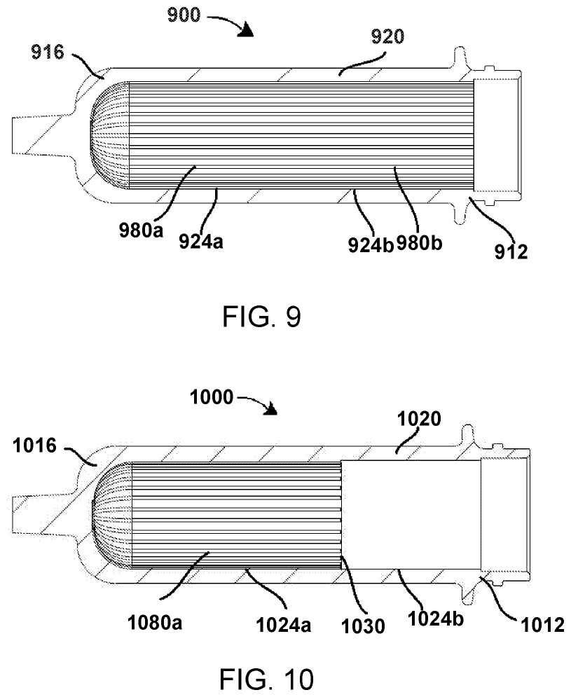

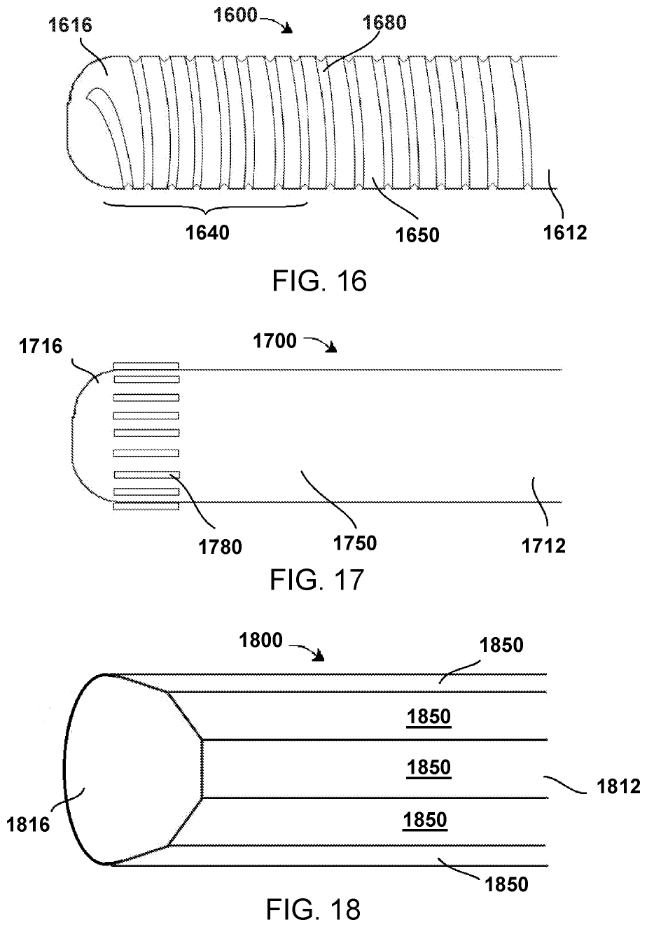

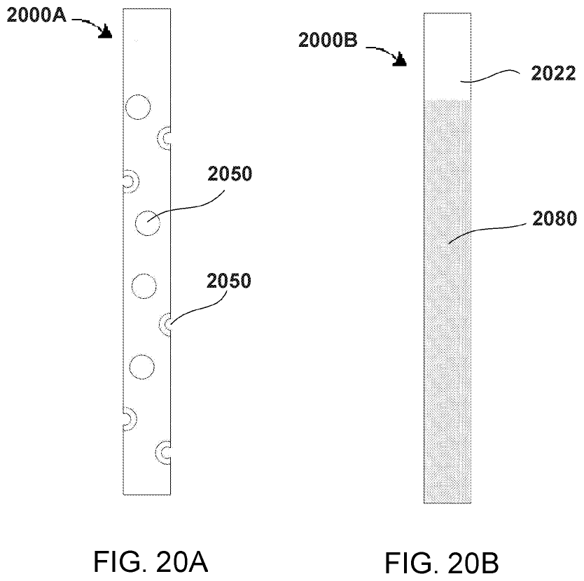

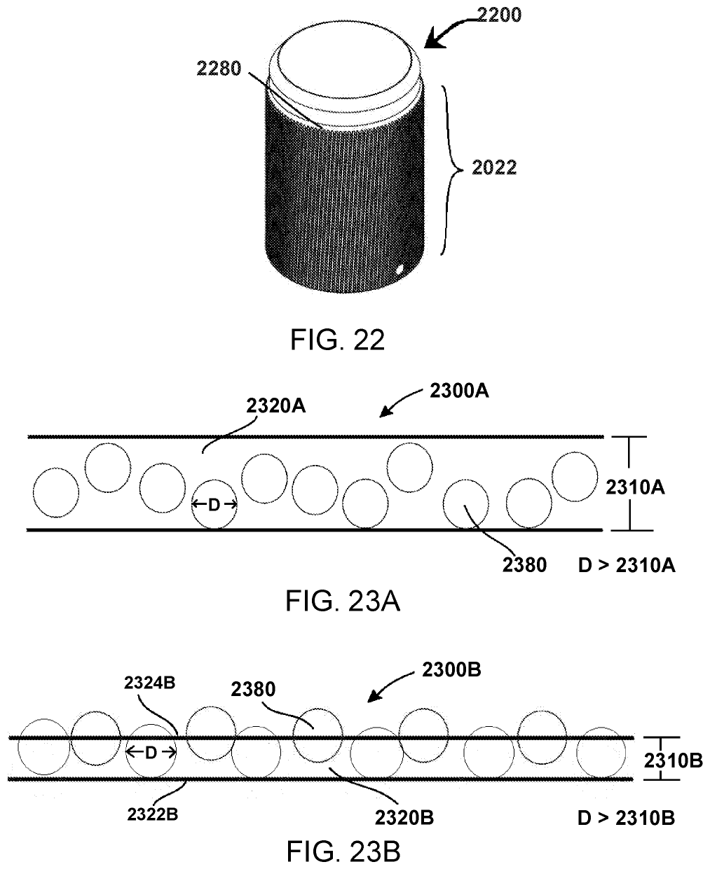

According to certain embodiments, a plurality of longitudinal ribs may be added to the inner wall of the preform (e.g., 900, 1000, or 1100 of FIGS. 9, 10, and 11, respectively) in the direction of the core draw for example by using a core 1500 (see FIG. 15) having a corresponding plurality of longitudinal grooves 1580 on at least a portion of the longitudinal axis thereof, or by using a core 1700 (see FIG. 17) that has features 1780 that may etch or scratch a plurality of grooves into the inner surface 824 of the preform 800, effectively providing a plurality of longitudinal ribs (regions between the grooves) along an axis thereof. By adding uniform or non-uniform longitudinal ribs 1580 along at least a portion of the core 1500, the core can be pulled out from the interior of the preform 900, 1000, or 1100 of FIGS. 9, 10, and 11, respectively, if the draft is sufficient. According to certain embodiments, a plurality of non-uniform longitudinal ribs may have an uneven depth along the longitudinal axis (see, FIGS. 19A and 19B), for example the plurality of non-uniform longitudinal preform ribs may be cut deeper into the core (i.e., the corresponding core grooves project radially farther inward in the part) corresponding the proximal end 812 of the preform 800 compared to the distal end 816 which will allow the core to be removed more readily. Alternatively, the plurality of non-uniform longitudinal preform ribs may be cut deeper into the core (i.e., the corresponding core grooves project radially farther inward in the part) corresponding the distal end 816 of the preform 800 compared to the proximal end 812 which will allow the core to be removed more readily. According to various embodiments, the uniform or non-uniform longitudinal ribs 980a, 980b may run over a majority of the longitudinal axis of the preform sidewall 920 (see, e.g., FIG. 9), or may run over only a portion, such as a proximal portion 1080a of the preform sidewall 1020 (see, e.g., FIG. 10). According to other embodiments, the uniform or non-uniform longitudinal ribs 1180 may extend for different lengths and/or at different regions along the longitudinal axis (see, e.g., FIG. 11). The uniform or non-uniform longitudinal ribs may be arranged in an even, repeating pattern around the circumference of the preform 800 or in other embodiments, the plurality of uniform or non-uniform longitudinal ribs may be arranged in a non-even pattern around the circumference of the preform 800. In certain embodiments, the plurality of non-uniform longitudinal ribs, as described herein, may have a varying width along the longitudinal axis, for example, the plurality of non-uniform longitudinal ribs may be wider at the proximal end comparted to the distal end or may be wider at the distal end compared to the proximal end. In other embodiments, the width of the plurality of longitudinal ribs may vary over a portion of or over the entire length of the longitudinal axis of the rib. The resulting preform 800 may have a plurality of longitudinal ribs with a non-uniform depth or width relative to the longitudinal axis. The longitudinal ribs of the preform 800 may be transferred to the inner surface 24 of the rolling diaphragm syringe 10 during a blow-molding process resulting in a syringe 10 having at least one surface texturization pattern comprising a uniform or non-uniform longitudinal rib on at least a portion of the inner surface 24 of the flexible sidewall 20 (see, e.g., FIGS. 4A to 5b).

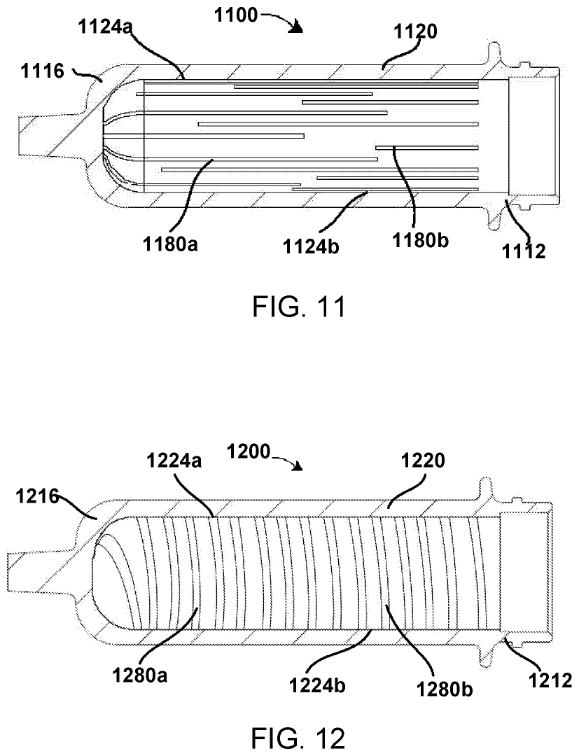

In another embodiment, the plurality of uniform or non-uniform ribs may have a clockwise or counterclockwise spiral or helical pattern along the longitudinal axis of the preform 1200 (see FIG. 12). According to certain embodiments, the spiral pattern may include a single uniform or non-uniform rib 1280 spiraling on the inner surface 1224 over at least a portion of the longitudinal axis of the preform 1200. According to other embodiments, the spiral pattern may include two or more uniform or non-uniform ribs 1280 spiraling on the inner surface 1224 over at least a portion of longitudinal axis of the preform 1200.

Various non-limiting embodiments of the rolling diaphragm syringes 10, the injection molded preforms 800, the structure of the core member for the injection molding process to form the preform 800, and processes to form embodiments of the at least one surface texturization feature will be described with reference to one or more of the accompanying drawings. The features of the syringes, preforms, and mold structures are not intended to be limited thereby.

With reference to FIGS. 4A and 4B, an embodiment of a rolling diaphragm syringe 410 having at least one surface texturization feature in the form of a plurality of longitudinal ribs 480 along a proximal portion of an inner surface 424a of a sidewall 420 thereof is illustrated. In this embodiment, the distal portion of the inner surface 424b of the sidewall 420 does not include the surface texturization feature. FIG. 4A illustrates the rolling diaphragm syringe 410 in the unrolled configuration. The proximal portion 416 of the syringe 410 has a plurality of uniform or non-uniform longitudinal ribs 480 along the inner surface 424a of the sidewall 420. The plurality of longitudinal ribs 480 may be of uniform depth and/or width according to certain embodiments. In other embodiments, the plurality of longitudinal ribs 480 may be of non-uniform depth and/or non-uniform width along the longitudinal axis. The plurality of longitudinal ribs 480 may be evenly distributed around the circumference of the proximal portion 416 of the sidewall 420 or in other embodiments may be unevenly distributed around the circumference of the proximal portion 416 of the sidewall 420 of the syringe 410. At least a portion of the distal portion 412 of the sidewall 420 may not have the longitudinal ribs 480 on the inner surface 424b thereof. FIG. 4B illustrates the rolling diaphragm syringe 410 having at least one surface texturization feature in the form of a plurality of longitudinal ribs 480 along a proximal portion of an inner surface 424a of a sidewall 420 in the rolled configuration. As can be seen, the majority of the length of the longitudinal ribs 480 on the proximal portion of the inner surface 424a of sidewall 420 are rolled to the interior of the rolled syringe while the distal portion of the inner surface 424b remains on the exterior of the rolled syringe. In certain embodiments, at least a portion of the surface texturization feature 480 on the proximal inner surface 424a may be transferred to distal inner surface 424b during the rolling process (not shown).

With reference to FIGS. 5A and 5B, an embodiment of a rolling diaphragm syringe 510 having at least one surface texturization feature in the form of a plurality of longitudinal ribs 580a, 580b along substantially the entirety of an inner surface 524a and 524b of a sidewall 520 thereof is illustrated. FIG. 5A illustrates the rolling diaphragm syringe 510 in the unrolled configuration. The entirety of the sidewall 520 of the syringe 510 has a plurality of uniform or non-uniform longitudinal ribs 580 along the inner surface 524a and 524b of the sidewall 520. The plurality of longitudinal ribs 580 may be of uniform depth and/or width according to certain embodiments. In other embodiments, the plurality of longitudinal ribs 580 may be of non-uniform depth and/or non-uniform width along the longitudinal axis. The plurality of longitudinal ribs 580 may be evenly distributed around the circumference of the sidewall 520 or in other embodiments may be unevenly distributed around the circumference of the sidewall 520 of the syringe 510. FIG. 5B illustrates the rolling diaphragm syringe 510 having at least one surface texturization feature in the form of a plurality of longitudinal ribs 580a, 580b along substantially the entirety of an inner surface 524a and 524b of a sidewall 520 in the rolled configuration. As can be seen, the plurality of surface texturization features span both the interior 580a and the exterior portion 580b of the inner surface 524a, 524b of the rolled sidewall 525 of the syringe 510.

According to certain embodiments, the plurality of ribbed surface texture features may extend to the proximal end wall 30 of the syringe 10. Adding ribbed texture lines to the base area of the syringe may increase the base inversion force (i.e., the force necessary to invert the contact end wall 30, for example during a retraction of the piston and end wall 30). Typically inversion of the concave end wall 30 is undesired as it results in error in volume and damage to the syringe which can potentially lead to creasing of the sidewall 20. The thickness and/or height of the textured rib features may vary directly with observed base inversion forces. According to other embodiments, a plurality of ribbed features may add additional beam strengthening to the flexible sidewall 20 to prevent volumetric shrinkage over time. For example, a plurality of longitudinal ribs may add longitudinal stiffness to the flexible sidewall 20. According to another example one or more spiral ribs around the circumference of the sidewall 20 may add stiffness to the wall to prevent volumetric shrinkage from bowing out or bowing in over time.

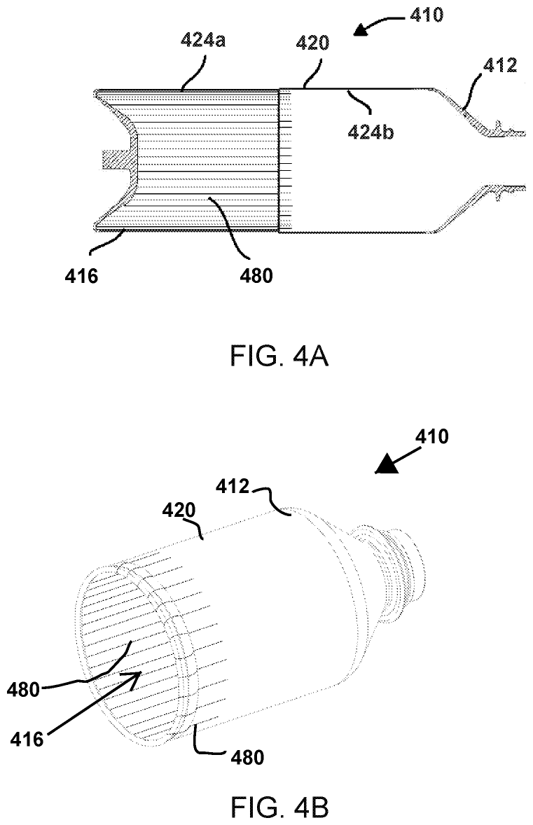

With reference to FIGS. 6A and 6B, an embodiment of a rolling diaphragm syringe 610 having at least one surface texturization feature in the form of one or more spiral ribs 680a, 680b along substantially the entirety of an inner surface 624a and 624b of a sidewall 620 thereof is illustrated. FIG. 6A illustrates an embodiment of the rolling diaphragm syringe 610 in the unrolled configuration. The entirety of the sidewall 620 of the syringe 610 has one or more uniform or non-uniform spiral ribs 680a, 680b along the inner surface 624a and 624b of the sidewall 620. The one or more spiral ribs 680a, 680b may be of uniform depth and/or width according to certain embodiments. In other embodiments, the one or more spiral ribs 680a, 680b may be of non-uniform depth and/or non-uniform width around the longitudinal axis. The one or more uniform or non-uniform spiral ribs 680a, 680b may be evenly distributed around the circumference of the sidewall 620 or in other embodiments may be unevenly distributed around the circumference of the sidewall 620 of the syringe 610 or may have different distances between successive turns. In certain embodiments, the spiral rib may only extend along a portion of the inner surface 680a of the sidewall 620 of the syringe 610. FIG. 6B illustrates the rolling diaphragm syringe 610 having one or more spiral ribs 680 in the rolled configuration. As can be seen, the plurality of surface texturization features span both the interior 680a and the exterior portion 680b of the inner surface 624a, 624b of the rolled sidewall 625 of the syringe 610. Due to the inversion of the proximal inner surface 624a relative to the distal inner surface 624b during the rolling process, the proximal one or more spiral ribs 680a may invert in direction relative to the distal one or more spiral ribs 680b leading to a cross-hatched patterns, which may reduce the observed coefficient of friction further due to a further reduction in surface to surface contact between the proximal inner surface 624a and the distal inner surface 624b.

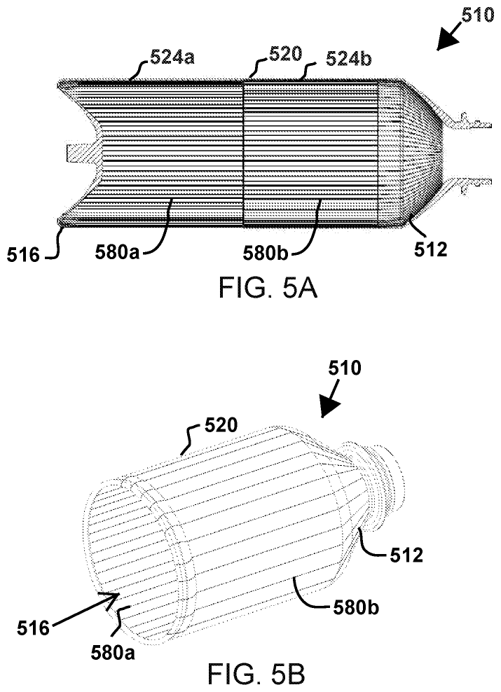

With reference to FIGS. 7A and 7B, an embodiment of a rolling diaphragm syringe 710 having at least one surface texturization feature in the form of a plurality of radially outward protruding or radially inward protruding dimples 780a, 780b substantially the entirety of an inner surface 724a and 724b of a sidewall 720 thereof is illustrated. In other embodiments, the dimpled at least one surface texturization feature 780 may be molded onto, for example in the form of proximal dimples 780a or distal dimples 780b, along only a portion the syringe 710. In certain embodiments, protruding dimples 780c protrude radially outward on the outer surface 722 of the sidewall 720 of unrolled syringe 710. In certain embodiments, the distal portion 712 of the inner surface 724b of the sidewall 720 may not include the dimpled surface texturization feature 780b. FIG. 7A illustrates an embodiment of the rolling diaphragm syringe 710 in the unrolled configuration. The entirety of the sidewall 720 of the syringe 710 displays a plurality of uniform or non-uniform dimple texturization features 780 along the inner surface 724a and 724b of the sidewall 720. The plurality of dimple texturization features 780 may be of uniform depth and/or radius according to certain embodiments. In other embodiments, the plurality of dimple texturization features 780 may be of non-uniform depth and/or non-uniform radius along the longitudinal axis. While circular dimples are illustrated, other shapes for the dimples are envisioned. The plurality of dimple texturization features 780 may be evenly distributed around the circumference of the sidewall 720 or in other embodiments may be unevenly distributed around the circumference of the sidewall 720 of the syringe 710. FIG. 7B illustrates the rolling diaphragm syringe 710 having at least one surface texturization feature in the form of a plurality of dimples 780 along a proximal portion of an inner surface 724a of a sidewall 720 in the rolled configuration. As can be seen in FIG. 7B, the dimples 780 project into the interior of the sidewall 720 of the syringe 710 providing a reduced area of surface to surface contact between portions of the inner surface 724a, 724b. According to certain embodiments, in the blow-molding process, the dimple texture features 780 may be molded so that they extend radially outward from the outer surface 722 of the sidewall 720. During the initial rolling process, the rolling apparatus may invert the direction of the dimples 780 so that they extend inwardly from the inner surface 724 of the sidewall due to the compression between as apparatus pressure jacket and a piston during rolling. Alternatively according to another embodiment, in the blow-molding process, the dimple texture features 780 may be molded so that they extend radially inward from the inner surface 724 of the sidewall 720. Inversion of the dimple feature 780 during a rolling process would not be necessary in this embodiment.

According to the various approaches described herein, the rolling diaphragm syringes having the at least one surface texturization feature thereon are typically formed via a stretch blow-molding process from an injection molded preform. In certain embodiments, at least one preform surface texturization feature may be initially molded one or imparted to the preform and then converted to the desired at least one surface texturization feature on an inner surface of the rolling diaphragm syringe. As the polymeric material expands and thins during the stretch blow-molding process, the size, depth, width, shape, relative position, and location of the at least one preform texturization feature must be selected to give the desired size, depth, width, shape, relative position, and location of the at least one texturization feature on the rolling diaphragm. The physical properties of the at least one surface texturization feature on the resulting syringe are chosen to provide the appropriate balance between having sufficient distance and low contact surface area between the distal and proximal regions of the inner surfaces of the sidewalls in the rolled configuration (resulting in undesired audible noise, entrapment of air between the inner surfaces, and strain on the injector motor) and not having too much distance where the sidewall and/or end wall portions of the syringe (increasing the chance that the syringe wall will buckle or crease during the rolling or unrolling process as the sidewalls move towards each other and overcome the hoop strength of the flexible sidewall). According to certain embodiments, the radial extension of the at least one surface texturization feature is selected so the distance between the inner surfaces of the sidewall of the syringe in the rolled configuration ranges from about 0.01 inches to about 0.06 inches. In other embodiments, the distance between the inner surfaces of the sidewall of the syringe in the rolled configuration ranges from about 0.02 inches to about 0.004 inches.

Various embodiments of the injection molded preform will now be described. With reference to FIGS. 8A and 8B, a preform 800 for blow-molding a rolling diaphragm syringe is illustrated without any surface texturization feature. The preform 800 has a sidewall 820 having an inner surface 824 and an outer surface 822. At the proximal end 816, the preform may have a molded piston engagement feature 830 to allow the resulting rolling diaphragm syringe to engage a piston of a fluid injector. At the distal end 812, the preform has a fluid outlet 840. During the stretch blow-molding process, the piston engagement feature 830 and the fluid outlet 840 at the distal end 812 are not significantly changed.

FIG. 9 illustrates an embodiment of the preform 900 having at least one preform surface texturization feature in the form of a plurality of preform longitudinal ribs 980a, 980b along substantially the entirety of an inner surface 924a and 924b of a sidewall 920 thereof. The plurality of preform longitudinal ribs 980a, 980b run substantially from the proximal end 916 to the distal end 912 of preform 900. During a stretch blow-molding process the plurality of preform longitudinal ribs 980a, 980b on the inner surface 924a and 924b of the sidewall 920 are stretched and blown to provide a rolling diaphragm syringe 510 as shown in FIG. 5A having a plurality of longitudinal ribs extending along the inner surface 524a, 524b of the syringe sidewall 520. The plurality of preform longitudinal ribs 980 may be of uniform depth, length, and/or width according to certain embodiments. In other embodiments, the plurality of preform longitudinal ribs 980 may be of non-uniform depth, non-uniform length, and/or non-uniform width along the longitudinal axis. The plurality of preform longitudinal ribs 980 may be evenly distributed around the circumference of the sidewall 920 or in other embodiments may be unevenly distributed around the circumference of the sidewall 920 of the preform 900. The plurality of longitudinal ribs 980a, 980b may be formed by using an injection molding core structure that either has longitudinal grooves or longitudinal ribs thereon (see FIG. 15). As described herein, the physical dimensions of the plurality of preform longitudinal ribs 980a, 980b may be selected to give the desired dimensional features of the longitudinal ribs 580 of the syringe 510.

FIG. 10 illustrates an embodiment of the preform 1000 having at least one preform surface texturization feature in the form of a plurality of longitudinal ribs 1080a along a proximal portion of an inner surface 1024a of a sidewall 1020 thereof. The plurality of longitudinal ribs 1080a run substantially from the proximal end 1016 to an intermediate point 1030 along the sidewall 1020 of the preform 1000. During a stretch blow-molding process the plurality of longitudinal ribs 1080a on the inner surface 1024a of the sidewall 1020 are stretched and blown to provide a rolling diaphragm syringe 410 as shown in FIG. 4A having a plurality of longitudinal ribs extending along a proximal portion of an inner surface 424a of the syringe sidewall 420. The plurality of preform longitudinal ribs 1080 may be of uniform depth, length, and/or width according to certain embodiments. In other embodiments, the plurality of preform longitudinal ribs 1080 may be of non-uniform depth, non-uniform length, and/or non-uniform width along the longitudinal axis. The plurality of preform longitudinal ribs 1080 may be evenly distributed around the circumference of the sidewall 1020 or in other embodiments may be unevenly distributed around the circumference of the sidewall 1020 of the preform 1000. The plurality of longitudinal ribs 1080a may be formed by using an injection molding core structure that either has longitudinal grooves or longitudinal ribs thereon. As described herein, the physical dimensions of the plurality of preform longitudinal ribs 1080a may be selected to give the desired dimensional features of the longitudinal ribs 480 of the syringe 410.

FIG. 11 illustrates an embodiment of the preform 1100 having at least one preform surface texturization feature in the form of a plurality of longitudinal ribs 1180 having differing lengths and/or positions along substantially the entirety of an inner surface 1124 of a sidewall 1120 thereof. The plurality of longitudinal ribs 1180 run substantially parallel to the longitudinal axis at different positions and for different lengths from the proximal end 1116 to the distal end 1112 of preform 1100. During a stretch blow-molding process the plurality of longitudinal ribs 1180 on the inner surface 1124 of the sidewall 1120 are stretched and blown to provide a rolling diaphragm syringe (not shown) having a plurality of longitudinal ribs dispersed along the inner surface of the syringe sidewall. The plurality of preform longitudinal ribs 1180 may be of uniform depth and/or width according to certain embodiments. In other embodiments, the plurality of preform longitudinal ribs 1180 may be of non-uniform depth, non-uniform width, and/or non-uniform width along the longitudinal axis. The plurality of preform longitudinal ribs 1180 may be evenly distributed around the circumference of the sidewall 1120 or in other embodiments may be unevenly distributed around the circumference of the sidewall 1120 of the preform 1100. The plurality of longitudinal ribs 1180 may be formed by using an injection molding core structure that either has longitudinal grooves or longitudinal ribs thereon. As described herein, the physical dimensions of the plurality of preform longitudinal ribs 1180 may be selected to give the desired dimensional features of the longitudinal ribs of the syringe.

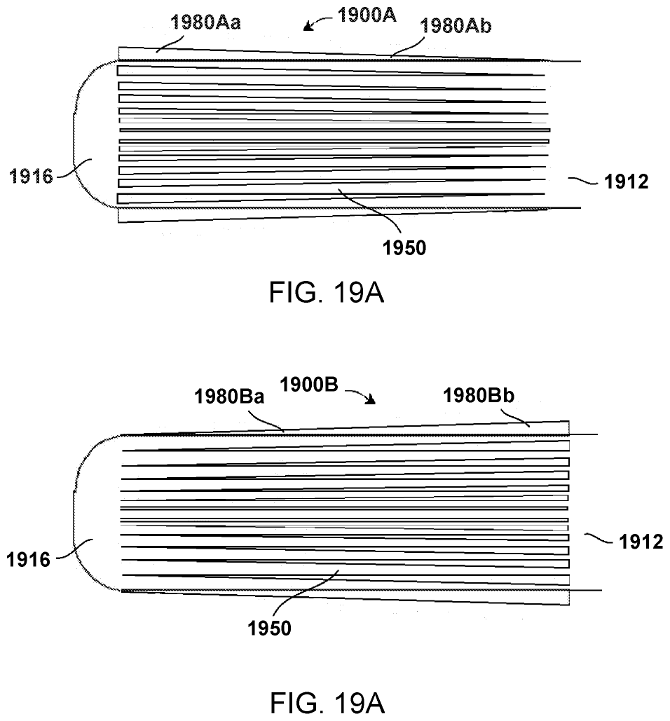

FIG. 12 illustrates an embodiment of the preform 1200 having at least one preform surface texturization feature in the form of one or more preform spiral ribs 1280a, 1280b along at least a portion of and in certain embodiments, the entirety of an inner surface 1224a and 1224b of a sidewall 1220 thereof. The one or more preform spiral ribs 1280a, 1280b may run from the proximal end 1216 to the distal end 1212 of preform 1200 or for any length in between. During a stretch blow-molding process the one or more preform spiral ribs 1280a, 1280b on the inner surface 1224a and 1224b of the sidewall 1220 are stretched and blown to provide a rolling diaphragm syringe 610 as shown in FIG. 6A having a one or more spiral ribs extending along the inner surface 624a, 624b of the syringe sidewall 620. The one or more preform spiral ribs 1280 may be of uniform depth, length, and/or width according to certain embodiments. In other embodiments, the one or more preform spiral ribs 1280 may be of non-uniform depth, non-uniform length, and/or non-uniform width around the longitudinal axis. The one or more preform spiral ribs 1280 may be evenly distributed around the circumference of the sidewall 1220 or in other embodiments may be unevenly distributed around the circumference of the sidewall 1220 of the preform 1200, including, for example, having different distances between adjacent spirals. The one or more preform spiral ribs 1280a, 1280b may be formed by using an injection molding core structure that either has spiral grooves or spiral ribs thereon (see FIG. 16) or may be formed during the stretch blow-molding process using a stretch rod, as described herein. As described herein, the physical dimensions of the one or more preform spiral ribs 1280a, 1280b may be selected to give the desired dimensional features of the one or more spiral ribs 680 of the syringe 610.

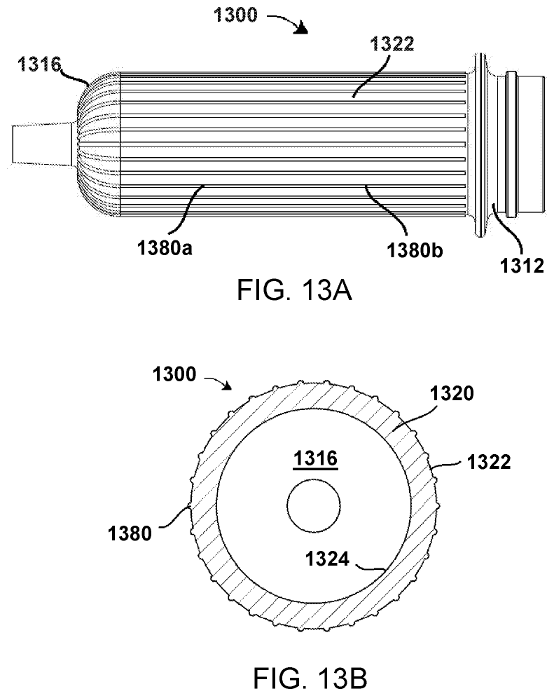

FIGS. 13A and 13B illustrate an embodiment of the preform 1300 having at least one preform surface texturization feature in the form of a plurality of preform longitudinal ribs 1380a, 1380b along an outer surface 1322a and 1322b of a sidewall 1320 thereof. The plurality of preform longitudinal ribs 1380a, 1380b run substantially from the proximal end 1316 to the distal end 1312 of preform 1300. During a stretch blow-molding process the plurality of preform longitudinal ribs 1380a, 1380b on the outer surface 1322 of the sidewall 1320 are stretched and blown to provide a rolling diaphragm syringe having a plurality of longitudinal ribs or surface texturization features 580 extending along the inner surface 524a, 524b of the syringe sidewall 520. The transfer of the preform longitudinal ribs 1380 from the outer surface 1322 of the preform 1300 to the inner surface 524 of the sidewall 520 of the syringe 510 may be affected during the blow-molding process. As the outer wall 1322 and preform longitudinal ribs 1380 are blown against the smooth mold wall the rib material is forced inward to provide the surface texturization feature 580 on the inner surface of the sidewall of the syringe 510. While the preform texturization feature is shown as longitudinal ribs 1380, any pattern or excess material on the outer surface 1322 of the preform 1300 may be utilized. For example, as shown herein, partial longitudinal ribs or spiral ribs may also be utilized to form at least one surface texturization feature on the inner surface 24 of the syringe 10. The plurality of preform longitudinal ribs 1380 may be of uniform height, length, and/or width according to certain embodiments. In other embodiments, the plurality of preform longitudinal ribs 1380 may be of non-uniform height, non-uniform length, and/or non-uniform width along the longitudinal axis. The plurality of preform longitudinal ribs 1380 may be evenly distributed around the circumference of the outer surface 1322 of the sidewall 1320 or in other embodiments may be unevenly distributed around the circumference of the outer surface 1322 of the sidewall 1320 of the preform 1300. The plurality of preform longitudinal ribs 1380 may be formed by using an injection molding outer mold structure that either has longitudinal grooves or longitudinal ribs thereon. As described herein, the physical dimensions of the plurality of preform longitudinal ribs 1380 may be selected to give the desired dimensional features of the longitudinal ribs 580 of the syringe 510.

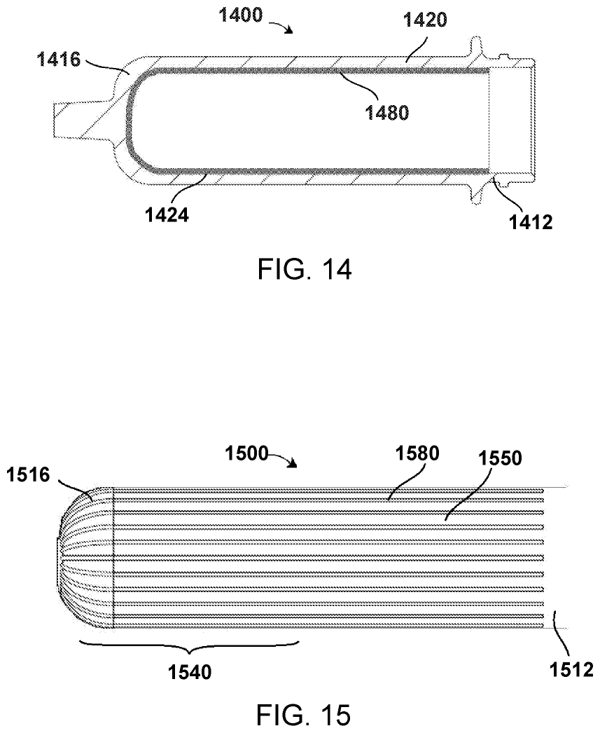

With reference to FIG. 14, a preform 1400 according to one embodiment is illustrated where the inner surface 1424 of the sidewall 1420 of the preform 1400 may have a coating of a material capable of providing at least one preform surface texturization feature 1480 on the inner surface 1424 of the sidewall 1420 of the preform 1400. For example, the coating material may be a molded in lubricating particulate or other lubricant material that has a lower coefficient of friction compared to the syringe polymeric material. In another example, the coating material may be a fibrous material applied from the surface of the injection molding core that is imbedded in the inner surface 1424 during molding. In certain embodiments, the fibers of the fibrous material may not melt or only partially melt at the mold temperature. The coating material may be applied during the injection molding process or after the process, directly to the preform 1400, for example, by spray application or dip application an may be coated on either the inner surface 1424 only or on both the inner surface 1424 and outer surface 1422. According to other embodiments, the material of the core may include a series of two or more laminate surfaces, including for example, a laminate on the inner surface 1424 of a polymeric material having a low coefficient of friction relative to the other sidewall materials. On example may be a laminate having PET as one outer layer and a polytetrafluoroethylene layer on the inner surface of the preform 1400. According to other embodiments, the inner surface 1424 of the preform may be treated with a surface texturization process, such as E-beam or plasma irradiation to form the at least one texturization feature on the inner surface 1424.

According to various embodiments, injection mold core members may also be used to apply the at least one surface texturization feature to an inner surface 824 of a sidewall 820 of a preform 800 during the injection molding process. Core surface texturization features may be directly transferred to the inner surface 824 of the preform sidewall 820 during the injection molding. With reference to FIG. 15, an injection mold core 1500 is illustrated according to various embodiments of the present disclosure. The injection mold core 1500 may have a plurality of uniform or non-uniform longitudinal ribs or grooves 1580 along the outer surface 1550 thereof. The plurality of longitudinal ribs or grooves 1580 may extend along at least a portion of the length of the core member 1500 from the proximal end 1516 to the distal end 1512 (with reference to the core member, proximal and distal nomenclature similar to that from the preform and/or syringe is used, i.e., the proximal or distal end of the core corresponds to the proximal or distal end of the preform and syringe, respectively). As shown in FIG. 15, the plurality of longitudinal ribs or grooves 1580 extend over the entire distance. In other embodiments, the plurality of longitudinal ribs or grooves 1580, may extend over only a portion, for example the proximal half 1540 of the core member 1500. The plurality of longitudinal ribs or grooves 1580 on the core member may be arranged to provide the corresponding uniform or non-uniform longitudinal ribs on the inner surface 824 of the preform 800 according to the various embodiments described herein.

With reference to FIG. 16, an injection mold core 1600 is illustrated according to various embodiments of the present disclosure. The injection mold core 1600 may have one or more uniform or non-uniform spiral ribs or grooves 1680 along the outer surface 1650 thereof. The one or more uniform or non-uniform spiral ribs or grooves 1680 may extend along at least a portion of the length of the core member 1600 from the proximal end 1616 to the distal end 1612. As shown in FIG. 16, the one or more spiral ribs or grooves 1680 extend over the entire distance from the proximal end 1616 to the distal end 1612. In other embodiments, the one or more spiral ribs or grooves 1680, may extend over only a portion, for example the proximal half 1640 of the core member 1600. The one or more spiral ribs or grooves 1680 on the core member may be arranged to provide the corresponding spiral ribs on the inner surface 824 of the preform 800 according to the various embodiments described herein.

With reference to FIG. 17, an injection mold core member 1700 is illustrated according to various embodiments of the present disclosure. The injection mold core 1700 may have a plurality of texture imparting features in the form of a plurality of etching protrusions 1780 extending along the outer surface 1750 thereof. The plurality of etching protrusions 1780 may be located at any position along the core member 1700. In one embodiment, as shown in FIG. 17, the plurality of etching protrusions 1780 are located near the proximal end 1716 of core member 1700. After an injection molding process, the core member 1700 is removed from the interior of the molded preform. As the core member 1700 is removed, the plurality of etching protrusions 1780 etch a corresponding plurality of uniform or non-uniform etched longitudinal grooves on an inner surface 824 of a sidewall 820 of the preform 800. The plurality of etched longitudinal grooves on the preform are similar to the uniform or non-uniform longitudinal ribs 980 illustrated in preform 900 of FIG. 9.

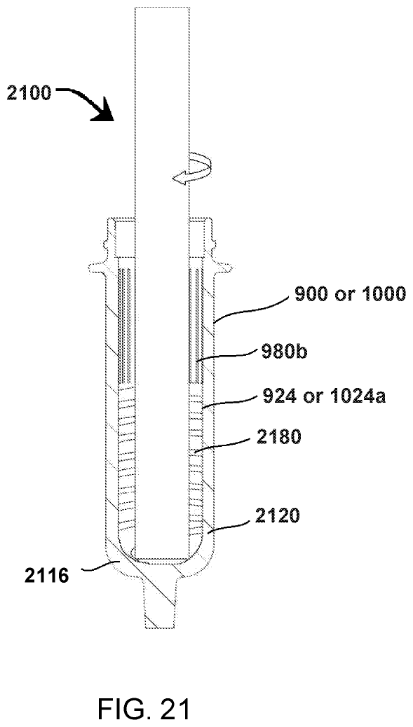

With reference to FIG. 18, an injection mold core member 1800 is illustrated according to various embodiments of the present disclosure. The injection mold core 1800 may have a polygonal cross-section and have a plurality of flat surfaces 1850 extending along the outer surface 1850 in the longitudinal direction. The flat surfaces 1850 may impart corresponding flat surface features on an inner surface 824 of a sidewall 820 of a preform 800 resulting in an inner surface 824 having a non-continuous polygonal cross-section. In embodiments where the number of flat surfaces 1850 is large, the resulting inner surface of the preform is molded to have a non-circular cross-section which may be transferred to the blow-molded syringe.

With reference to FIGS. 19A and 19b, two embodiments of an injection mold core 1900A and 1900B are illustrated according to various embodiments of the present disclosure. The injection mold core 1900A and 1900B may have a plurality of non-uniform longitudinal ribs or grooves 1980A and 1980B along the outer surface 1950 thereof. The height of the ribs or the depth of the grooves may be dependent on a variety of factors and may be chosen to provide the best combination of reduction of the coefficient of friction and/or air release and ease of core removal. The plurality of longitudinal ribs or grooves 1980A and 1980B may extend along at least a portion of the length of the core member 1900 from the proximal end 1916 to the distal end 1912. As shown in FIGS. 19A and 19b, the plurality of non-uniform longitudinal ribs or grooves 1980A and 1980B may extend over the entire length of the core sidewall and have a non-uniform height (ribs) or depth (grooves). FIG. 19A illustrates an embodiment where the non-uniform longitudinal ribs or grooves 1980A are of greater height or depth near the proximal end 1916 and ramp to a lower height/depth near the distal end 1912. FIG. 19B illustrates an embodiment where the non-uniform longitudinal ribs or grooves 1980B are of greater height or depth near the distal end 1912 and ramp to a lower height/depth near the proximal end 1916. In other embodiments, the plurality of longitudinal ribs or grooves 1980A and 1980B, may extend over only a portion, for example the proximal half 1940 of the core member 1900. In other embodiments, the plurality of longitudinal ribs or grooves 1980A and 1980B, may have a non-uniform width, for example the proximal end 1916 relative to the distal end 1912. In other embodiments, the plurality of non-uniform longitudinal ribs or grooves 1980A and 1980B may vary in heights/depth and width over the length of the core member 1900. The plurality of longitudinal ribs or grooves 1980A and 1980B on the core member may be arranged to provide the corresponding non-uniform longitudinal ribs on the inner surface 824 of the preform 800 according to the various embodiments described herein.

According to still other embodiments, the core member may include at least one core member surface texturization feature such as a non-uniform surface that may be imparted to the preform 800 during an injection molding process. Considerations regarding the amount of draft necessary to remove the core member may be necessary. In certain embodiments, the core member may include a plurality of core member surface texturization features that may extend from an outer surface of the core during the injection molding but may be retractable to allow for removal of the core member from the interior of the formed preform.

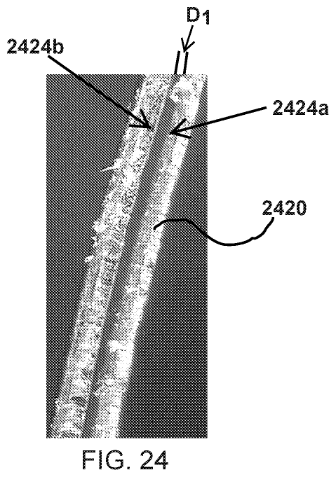

According to still other embodiments, the plurality of surface texturization feature may be applied during the stretch blow-molding process, for example using the stretch rod to impart the at least one surface texturization feature. Referring to FIG. 20A, an embodiment of a stretch rod 2000A suitable for use to impart the at least one surface texturization feature is illustrated. The stretch rod 2000A may be formed as a hollow tube with a plurality of openings 2050 along the longitudinal axis thereof. During the stretch blow-molding process the stretch rod 2000A may be equipped with an apparatus for ejecting particulates through the plurality of openings 2050 onto an inner surface 24 of the syringe 10. As the particulates strike the inner surface 24 of the syringe 10, the impact creates a plurality of surface texturization features on the inner surface 24 of the sidewall 20 with a “sand-blasting” type action. Suitable particulates may include particles that may be readily removed such as sand, polymeric beads; particulates that evaporate such as dry-ice, ice; and particulates that may be incorporated or dissolved into the injected solution, such as solid contrast agent particles, sodium chloride crystals, solid particles of the appropriate medicament, particulate silicone, and the like. In certain embodiments, a vacuum may be applied to the hollow tube of the stretch rod 2000A after particulate blasting to remove the particulates.

Referring to FIG. 20B, an embodiment of a stretch rod 2000B suitable for use to impart the at least one surface texturization feature is illustrated. The stretch rod 2000B may be formed as a having a plurality of texture imparting features 2080 along the outer surface 2022 thereof. During the stretch blow-molding process the stretch rod 2000B may be contacted on at least a portion of an inner surface 24 of the syringe 10. In certain embodiments, the plurality of texture imparting features 2080 may include a roughened outer surface 2022. According to other embodiments, the plurality of texture imparting features 2080 may be on an extending mechanism, such as a flexible surface or a brush-like fiber that may move radially outward, for example when the stretch rod 2000B is rotated, to contact at least a portion of the inner surface 22 of the sidewall 20 of the syringe 10. Upon contacting, the plurality of texture imparting features 2080 may impart at least one surface texturization feature on the inner surface 22 of the sidewall 20, for example a series of uniform or non-uniform etches or scratches.

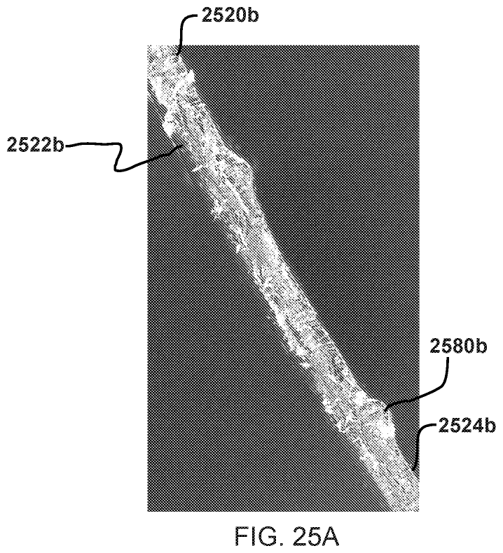

Referring to FIG. 21, an embodiment of a stretch rod 2100 suitable for use to impart a plurality of spiral surface texturization features is illustrated. According to this embodiment, a preform 900 (such as described with reference to FIG. 9) or preform 1000 (see FIG. 10) having at least one preform surface texturization feature in the form of a plurality of preform longitudinal ribs 980a, 980b, 1080a along substantially the entirety of or a proximal portion of an inner surface 924a, 924b, 1024a of a sidewall 920, 1020 thereof, respectively. The plurality of preform longitudinal ribs 980a, 980b, 1080a run substantially from the proximal end 916 to the distal end 912 of preform 900 or from the proximal end 1016 to a central portion 1030 of the preform 1000. According to these embodiments during the stretch blow-molding process, the stretch rod 2100 may be placed against the proximal end 2116 during stretch blow-molding and vibrated or rotated at one or more different rotational speeds and/or directions. As the preform 900, 1000 is stretched and blown, vibration or rotation of the stretch rod 2100 while proximally stretching results in a plurality of non-uniform, non-longitudinal ribs 2180 along the resulting inner surface 924a, 924b, 1024a of the flexible sidewall 2120 of the syringe. A benefit of this method is that a rolling diaphragm syringe having a plurality of spiral surface texturization features 2180 similar to the syringe illustrated in FIGS. 6A and 6B, without the difficulty associated with removal of a core member 1600 having one or more spiral ribs or grooves 1680, as illustrated in FIG. 16, from the preform 1200.

In other embodiments, the plurality of surface texturization features on the inner surface of the rolling diaphragm may be imparted using a textured plunger in an initial rolling process. As described herein after blow-molding of the syringe, the end wall 30 of the syringe 10 (see, e.g., FIG. 1A) may be rolled to the rolled configuration (see, e.g., FIG. 2A). During this process, a plunger is inserted into a concave portion of the end wall 30 and moved in a distal direction to roll the flexible sidewall 20 in on itself. With reference to FIG. 22 according to these embodiments, providing a surface texture 2280 on an outer wall 2022 of the roll plunger 2200 and then using to roll a rolling diaphragm syringe 10 in a rolling apparatus, the surface texture 2280 is imparted to an inner surface 24a of the rolled rolling diaphragm syringe. According to certain embodiments, the roll plunger 2200 and/or the inner surface of the pressure jacket of the roll apparatus (not shown) may be heated, for example at at temperature from room temperature up to the glass transition temperature Tg of the material, to facilitate transfer of the surface texture 2280 to the sidewall 20. Since the roll plunger 2200 contacts primarily the proximal end 16 and proximal outer wall 22a of the syringe, the surface texture 2280 of the roll plunger 2200 is transferred to the proximal inner wall 24a of the syringe 10. Various texturized features, such as longitudinal ribs, lateral ribs, diagonal/spiral ribs, random surface texture, specific graphical features, such as words, logos, and outlines, may be utilized at the surface texture 2280 of the roll plunger 2200.

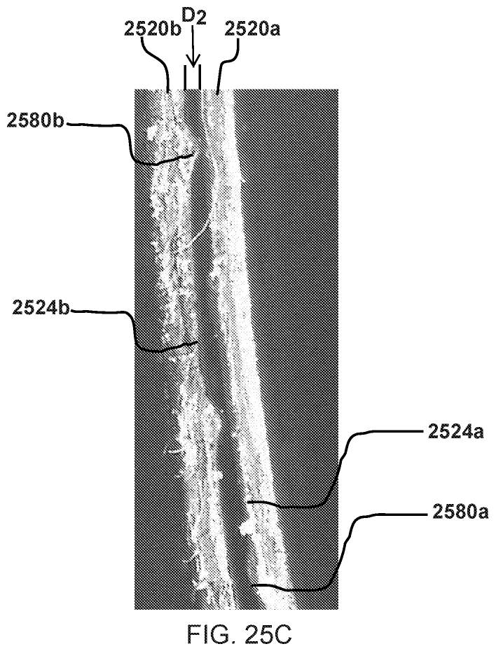

According to other embodiments, the preform may be molded with a plurality of fibers or particles formed from a material that has a higher glass transition temperature Tg than the preform polymer. As illustrated in FIGS. 23A and 23B, the fibers or particles 2080 may be embedded in the polymeric material of the side wall 2320A of the preform 2300. The particles or fibers 2380 are selected to have the necessary chemical and physical properties and size to have diameters D less than the width 2310A of the preform sidewall 2320A but greater than the width 2310B of the syringe sidewall 2320B. As the preform is stretch blow-molded and the width of the sidewall decreases, the fibers or particles 2080 extend outward from the sidewall 2320B providing the plurality of surface texturization features for the rolling diaphragm syringe. In other embodiment, the syringe polymer material may include embedded fiber or particulate material that results in texturization during a rolling process. According to other embodiments, the fibers or particulate materials may have a lower coefficient of friction compared to the sidewall material to provide a series of protruding surfaces on the inner surface 2324B of the sidewall 2320B. For example, a plurality of polymer beads 2080 having a higher glass transition temperature Tg than the syringe wall material may be embedded into the syringe polymer material. After rolling, the polymer fiber or particles 2080 may create non-uniformities and texture on the surface of the inner wall of the rolling diaphragm syringe. The polymer fiber or particles 2080 may have a diameter substantially similar to, greater than, or less than the width of the blow-molded syringe wall

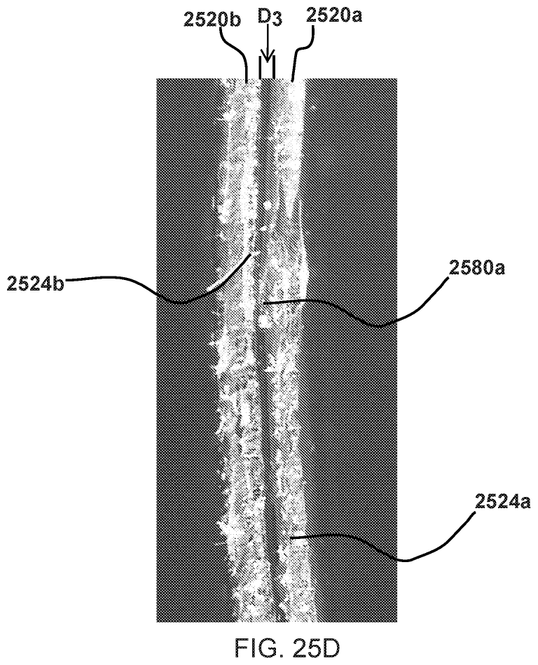

As described herein, the distance between the distal and proximal portions of the inner sidewall of the syringe may determine, at least in part, the coefficient of friction (μ) between the two sidewall surfaces. For example, with reference to FIG. 24 which is a microscope cross-sectional image of the contact area of two side wall portions 2424a and 2424b of a syringe, if the distance D₁ is too small, the coefficient of friction (μ) becomes large resulting in undesired audible squeaks as the inner sidewall slide relative to one another during rolling or unrolling, and may put strain on a motor of the medical injector as it moves the piston. Further, air may become entrapped between the inner surfaces 2424a and 2424b of the side wall 2420.

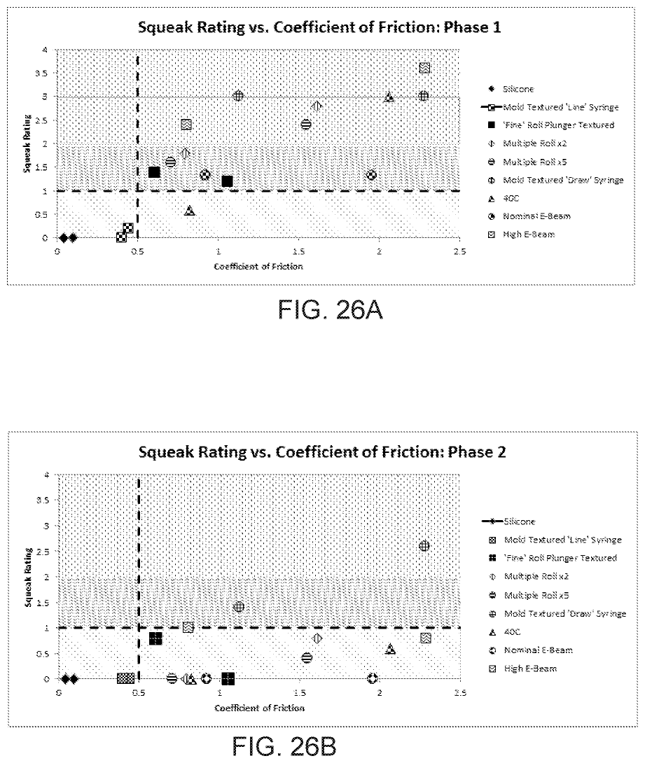

Referring now to FIGS. 25A to 25D, microscope cross-sectional images of the contact area of two side wall portions 2524b and 2524a are shown. With reference to FIG. 25A, a cross-sectional image of an outer surface 2522b and an inner surface 2524b of a distal sidewall portion 2520b is shown having a plurality of surface texturization features 2580b on an inner surface 2524b of the sidewall 2520b. Similarly, FIG. 25B, a cross-sectional image of an outer surface 2522a and an inner surface 2524a of a proximal sidewall portion 2520a is shown having a plurality of surface texturization features 2580a on an inner surface 2524a of the sidewall 2520a. In certain embodiments, the height of the surface texturization feature 2580a at the proximal sidewall portion 2520a is smaller than that of texturization feature 2580b at the distal sidewall portion 2520b. This may be due to the pressure imparted onto the proximal sidewall portion 2520a during the rolling process in the rolling apparatus.

With reference to FIG. 25C, a cross-sectional image showing a region of contact between a distal sidewall portion 2520b and a proximal sidewall portion 2520a of an embodiment is shown. The surface texturization features 2580a and 2580b on the inner surface 2524a and 2524b, respectively are directed towards each other and maintain a distance D₂ between the distal sidewall portion 2520b and a proximal sidewall portion 2520a, reducing the coefficient of friction (μ) and providing fluid paths for release of entrapped air. The distance D₂ between the distal sidewall portion 2520b and a proximal sidewall portion 2520a is greater than distance D₁ from the non-textured inner surfaces shown in FIG. 24. According to an embodiment shown in FIG. 25C, the distal inner surface 2524b has ribs (“unrolled inner ribs”) that are of approximately 0.003 inches and proximal inner surface 2524a has ribs (“rolled inner ribs”) that are approximately 0.0015 inches (smaller due to compression during rolling process). According to this embodiment, the distance between the distal sidewall portion 2520b and a proximal sidewall portion 2520a may range from 0.0015 inches to up to 0.005. The cross-section image shown in FIG. 25C would be indicative of a cross-section observed in the rolled syringe 510 shown in FIG. 5B.

With reference to FIG. 25D, a cross-sectional image showing a region of contact between a distal sidewall portion 2520b and a proximal sidewall portion 2520a of an another embodiment is shown. The surface texturization features 2580a are only imparted to the inner surface 2524a of the proximal portion of the sidewall 2520a of the syringe while the inner surface 2524b of the distal portion of the sidewall 2520b is substantially untextured. The surface texturization feature maintains a distance Da between the distal sidewall portion 2520b and a proximal sidewall portion 2520a, reducing the coefficient of friction (μ) and providing fluid paths for release of entrapped air. The distance Da between the distal sidewall portion 2520b and a proximal sidewall portion 2520a is greater than distance D₁ from the non-textured inner surfaces shown in FIG. 24 but less than the distance D2 from the textured inner surfaces shown in FIG. 25C.

### EXPERIMENTAL SECTION

The following variables were examined and exhibited positive impact on the coefficient of friction (μ), audible squeak, and entrapped air reduction results. The rolling/unrolling process was broken into three phases: Phase 1 includes initial retraction of the plunger from 0 mL to about 60 mL; Phase 2 includes movement of the piston distally from 60 mL to about 10 mL to purge air from the syringe; and Phase 3 includes proximal retraction of the plunger from 10 mL to 150 mL to provide a fully filled syringe. Variables that exhibited no significant positive effect are not discussed.

Silicone—Silicone exhibited a significant improvement across all three categories including friction, squeak, and air entrapment results. Using silicone, the friction results were negligible, the squeak was non-existent, and the air rating showed significant improvement at approximately half of the baseline level. However, introduction of silicone into the interior of the syringe may require additional testing and regulatory approval processes.

Mold Textured ‘Line’ Syringe—According to this variable, longitudinal ‘line’ texturization features were molded onto an inner surface of the syringe, such as in the embodiments described in FIGS. 4A to 6B. Testing of these surface texturization feature showed a near eliminated squeak with only one sample showing very minor squeak. These syringes also showed an improvement in air presence removal.

‘Fine’ Roll Plunger Textured—Syringes rolled with the textured plunger saw a significant reduction in coefficient of friction (μ) and squeak effects. Minimal effect on the air rating (air entrapment) was observed.

Multiple Roll—The squeak exhibited an improvement with the multiple rolls during the first two phases but was significantly worse in Phase 3.

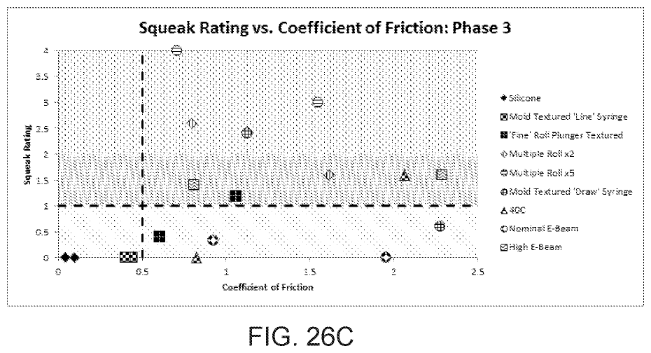

FIGS. 26A, 26B, and 26C provide a correlation of the relationship observed between friction and resulting audible squeak. Generally, the lower coefficient of friction (μ) indicates a reduced squeak rating. As seen in graphical results, silicone and the mold applied texture ‘line’ syringes are consistently below this acceptance threshold across all three phases of fluid delivery.

### Purpose

The purpose of this study was to isolate and analyze the variables that are considered potential contributing factors to squeak and air issues experienced during the rolling diaphragm syringe fill and delivery process. Variables or combinations of variables exhibiting the largest contribution of improvement will be implemented as applicable into the future Tool design. For each parameter, both PET material grade MN021 (93/280° F.) and MN052 (93/270° F.) syringes were evaluated for friction as well as fill and delivery performance. A standard baseline scenario was used to compare against each of the variations listed below.

### Variables

Baseline: The baseline used for comparison includes a single roll with a standard smooth roll plunger with diameter of 1.835″ and a typical aging process of 60° C. for 16 hours which emulates changes due to shipping and storage conditions.

Silicone: A thin layer of silicone was applied to the inside of each syringe before rolling and aging per the standard baseline format.

Temperature Comparison: Syringes were rolled per the baseline format but were exposed to 40° C. for 24 instead of 60° C. for 16 hours.

Multiple Roll: Syringes were rolled multiple times, 2× and 5×, in comparison to the baseline single roll before aging per the baseline format.

Sterilization: Syringes were rolled per the baseline format and then sterilized with nominal and high E-Beam levels and aged per the baseline format.

Syringe Texture: Syringes with a fine ‘draw’ texture (i.e., smooth inner surface with no texturization features) and a ‘line’ texture that were imparted via the molding process and then rolled and aged per the baseline format.

Plunger Texture: A textured plunger seen in FIG. 22 was used to roll the syringes before aging per the baseline format.

I. Friction Test

### Setup

Friction was evaluated for each variable listed above using a digital force gauge to calculate the peak tensile force as a result of sliding apart the two contacting surfaces. Two pieces of materials were laid on top of each other mimicking the contact they would have during fill. The top piece was attached to the force gauge while ensuring that the connection piece was maintained level and the bottom material attached to the base in order to remain in place. A weight of specified amount was placed on top of the layered materials in order to ensure contact. See FIGS. 6 and 7 below for test setup.

### Friction Test Results

TABLE 1
Coefficient of Friction Results by Variable

| | | Coefficient of | | Coefficient of | |
|---|---|---|---|---|---|
| | | Friction | | Friction | |
| | | (MN021 | | (MN052 | |
| | | 93/280) | StDev | 93/280) | StDev |
| Baseline | | 2.07 | 0.22 | 0.81 | 0.07 |
| Silicone | | 0.10 | 0.12 | 0.04 | 0.06 |
| Sterilization | Nominal E- | 1.95 | 0.43 | 0.92 | 0.10 |
| | Beam | | | | |
| | High E-Beam | 2.29 | 0.21 | 0.81 | 0.12 |
| Multiple | 2x | 1.61 | 0.21 | 0.79 | 0.10 |
| Roll | 5x | 1.55 | 0.04 | 0.71 | 0.02 |
| Textured Plunger | | 1.06 | 0.30 | 0.60 | 0.06 |
| Textured | No Texture | 1.47 | 0.27 | 1.69⁽¹⁾ | 0.19 |
| Syringe⁽²⁾ | Draw | 2.28 | 0.37 | 1.13⁽¹⁾ | 0.36 |
| | Line | 0.44 | 0.02 | 0.40⁽¹⁾ | 0.02 |
| 40° C. | | 2.06 | 0.30 | 0.83 | 0.04 |

Note⁽¹⁾:
Samples were of 93/270 mold temperature
Note⁽²⁾:
Samples were from 12-17 batch of syringes. A separate baseline was established for these specific syringes.
Note⁽³⁾:
The coefficient of friction was calculated as follows based on the mass of the applied weight (5.543 lbf) and the measured peak force:
$Coefficient of Friction ( μ ) = \frac{Peak Force}{Mass of Weight}$

### Fill Performance: Squeak

### Test Setup

Syringes of each variable listed above were tested per the Fill Performance. In each of the phases noted in this procedure, the effect of squeak considered both the duration of the squeaking as well as the intensity and developed as follows:

### Squeak Scoring System

Duration: N/A (0), Short (1), Long (2)
Intensity: N/A (0), Quiet (1), Loud (2)
Duration and Intensity results were multiplied to get a factor of contribution (0, 1, 2, or 4) and like samples averaged to produce below results

TABLE 2
Squeak Rating by Variable (0: Best-4: Worst)

| | | MN021 | | | MN052 | | |
|---|---|---|---|---|---|---|---|
| | | Phase I | Phase II | Phase III | Phase I | Phase II | Phase III |
| | | (0-60 | (60-10 | (10-150 | (0-60 | (60-10 | (10-150 |
| | | ml) | ml) | ml) | ml) | ml) | ml) |
| Baseline | | 4.0 | 1.4 | 2.4 | 3.0 | 1.0 | 1.8 |
| Silicone | | 0.0 | 0.0 | 0.0 | 0.0 | 0.0 | 0.0 |
| Sterilization | Nominal E- | 4.0 | 1.4 | 2.8 | 3.6 | 1.8 | 2.8 |
| | Beam | | | | | | |
| | High E- | 3.6 | 0.8 | 1.6 | 2.4 | 1.0 | 1.4 |
| | Beam | | | | | | |
| Multiple | 2x | 2.8 | 0.8 | 1.6 | 1.8 | 0.0 | 2.6 |
| Roll | 5x | 2.4 | 0.4 | 3.0 | 1.6 | 0.0 | 4.0 |
| Plunger Texture | | 1.2 | 0.0 | 1.2 | 1.4 | 0.8 | 0.4 |
| Textured | No Texture | 3.6 | 1.6 | 2.0 | 4.0 | 3.2 | 3.6 |
| Syringe⁽¹⁾ | Draw | 3.0 | 2.6 | 0.6 | 3.0 | 1.4 | 2.4 |
| | Texture | | | | | | |
| | Line Texture | 0.2 | 0.0 | 0.0 | 0.0 | 0.0 | 0.0 |

| | | | | | | |
|---|---|---|---|---|---|---|
| 40° C. | 3.0 | 0.6 | 1.6 | 0.6 | 0.0 | 0.0 |

Note⁽¹⁾:
Samples were from 12-17 batch of syringes. A separate ‘No Texture’ baseline was established for these specific syringes.
Note (2):
Values in BOLD show the variables with significant improvement.

### FN Performance: Air

### Test Setup

Syringes of each variable listed above were tested per the Fill Performance. In each of the phases noted in this procedure, air effects were evaluated considering the location of the air bubbles and the amount of combined surface area. An air rating was developed as follows:

### Air Scoring System

Location: N/A (0), Sidewall (1), Flare (2)
Surface area: N/A (0), Small (1), Large (2)
Location and Surface Area results were multiplied to get a factor of contribution (0, 1, 2, or 4) and like samples averaged to produce below results

TABLE 3
Air Rating by Variable (0: Best-4: Worst)

| | | MN021 | | | MN052 | | |
|---|---|---|---|---|---|---|---|
| | | Phase I | Phase II | Phase III | Phase I | Phase II | Phase III |
| | | (0-60 | (60-10 | (10-150 | (0-60 | (60-10 | (10-150 |
| | | ml) | ml) | ml) | ml) | ml) | ml) |
| Baseline | | 4.0 | 4.0 | 4.0 | 4.0 | 3.6 | 3.6 |
| Silicone | | 2.4 | 2.4 | 1.6 | 2.0 | 2.2 | 2.0 |
| Sterilization | Nominal E- | 4.0 | 4.0 | 4.0 | 4.0 | 4.0 | 4.0 |
| | Beam | | | | | | |
| | High E-Beam | 4.0 | 4.0 | 4.0 | 3.2 | 3.2 | 3.2 |
| Multiple | 2x | 3.6 | 2.6 | 2.4 | 3.2 | 2.2 | 2.0 |
| Roll | 5x | 3.2 | 2.8 | 1.6 | 3.2 | 3.0 | 2.8 |
| Plunger Texture | | 4.0 | 4.0 | 4.0 | 3.6 | 3.6 | 3.6 |
| Textured | No Texture | 4.0 | 4.0 | 4.0 | 4.0 | 4.0 | 4.0 |
| Syringe | ‘Draw’ | 4.0 | 3.6 | 3.6 | 4.0 | 3.6 | 3.6 |
| | Texture | | | | | | |
| | ‘Line’ | 2.6 | 2.6 | 2.4 | 2.2 | 2.2 | 1.8 |
| | Texture | | | | | | |

| | | | | | | |
|---|---|---|---|---|---|---|
| 40° C. | 2.8 | 3.2 | 3.2 | 2.8 | 2.8 | 2.8 |

Note (1):
Values in BOLD show the variables with significant improvement.

Air ratings for syringes with silicone applied compared to the baseline. The air was consistently less with the silicone.

The multiple rolled syringes showed a slight improvement of the air rating across all phases and both material types; however, the MN052 syringes showed a similar or worse impact on the 5× roll syringes compared to the 2× roll across each phase.

The ‘Line’ textured syringe showed substantial improvement while the ‘draw’ textured syringe showed very minimal improvement in air presence.

The 40° C. syringes had an improved effect on presence of air in each phase and material type.

### Conclusions for PET Materials

Silicone: Silicone showed a significant improvement across all three categories including squeak, friction and air results. The squeak was non-existent, the friction results were negligible and the air rating showed significant improvement at approximately half of the baseline level.

Sterilization: Sterilization showed no impact to the friction results. There was a minimal improvement on the air effects. The squeak showed no improvement between the baseline and nominal sterilization level in MN021 syringes and was slightly worse in the MN052 syringes. However, the High E-Beam level did show a slight improvement across both material types and each of the three phases.

Multiple Roll: The multiple rolled MN021 syringes showed a decrease in friction; however the MN052 syringes that have a lower baseline showed minimal change. There was not a significant improvement between to 2× and 5× rolled syringes. There was also an improvement in the air rating seen in the multiple rolled syringes versus the baseline although there was not a noticeable trend between the 2× and 5× syringes. The squeak showed an improvement with the multiple rolls during the first two phases but was significantly worse in Phase 3.

Textured Plunger: Syringes rolled with the textured plunger saw a significant reduction in coefficient of friction and squeak effects. There was no effect on the air rating.

Textured Syringes: The ‘line’ texture syringe nearly eliminated squeak with only one sample showing very minor squeak. These syringes also showed an improvement in air presence; however this was still not eliminated. The ‘draw’ texture syringes showed minimal improvement in both squeak and air.

40° C.: The syringes exposed to 40° C. rather than the 60° C. baseline resulted in no difference in friction. There was a significant reduction in squeak showing a very low rating in all phases and materials with exception of the MN021 syringes in Phase 1 which still saw an improvement. There was also an improvement in air presence. Although not as significant as with squeak, the air rating was consistently lower across each phase and material type.

It can be seen that certain variables had an impact, and although air effects were not eliminated by any sole parameter, squeak was eliminated with the use of both silicone and the ‘line’ textured syringes. Since silicone is not a preferred solution at this time, these ‘line’ textured syringes will serve to address the squeak issue moving forward.

It should be noted that the various aspects and embodiments of the present disclosure, while focused on the application to a rolling diaphragm syringe, may have application to any field that utilizes rolling diaphragm apparatuses for retaining and delivering a fluid.

While aspects of a rolling diaphragm syringe having a texturized inner surface and method of texturizing an inner surface of a rolling diaphragm syringe are provided in the foregoing description, those skilled in the art may make modifications and alterations to these aspects without departing from the scope and spirit of the disclosure. Accordingly, the foregoing description is intended to be illustrative rather than restrictive. The disclosure described hereinabove is defined by the appended claims and all changes to the disclosure that fall within the meaning and the range of equivalency of the claims are to be embraced within their scope.

1. A rolling diaphragm syringe for receiving a medical fluid therein, the rolling diaphragm syringe comprising:
a closed proximal end wall for releasably engaging a piston of a fluid injector;
a distal end having a neck and a fluid outlet;
a flexible sidewall extending between the proximal end wall and the distal end, wherein the flexible sidewall rolls upon itself when acted upon by the piston such that the outer surface of the flexible sidewall rolls in a radially inward direction as the piston is advanced from the proximal end to the distal end and unrolls in a radially outward direction as the piston is retracted from the distal end to the proximal end; and
at least one surface texturization feature on at least a portion of an inner surface of the flexible sidewall.

2. The rolling diaphragm syringe of claim 1, wherein the rolling diaphragm syringe is made from a medical grade polyethylene terephthalate (PET).

3. The rolling diaphragm syringe of claim 1, wherein the at least one surface texturization feature reduces a coefficient of friction (μ) between contacting portions of the inner surface of the flexible sidewall as it is rolled or unrolled.

4. The rolling diaphragm syringe of claim 3, wherein the coefficient of friction (μ) between contacting portions of the inner surface of the flexible sidewall having the at least one surface texturization feature as the flexible sidewall is rolled or unrolled ranges from 0.10 to 2.30.

5. The rolling diaphragm syringe of claim 1, wherein the at least one surface texturization feature is selected from the group consisting of a plurality of uniform or non-uniform longitudinal ribs, a plurality of uniform or non-uniform ribs having a spiral configuration around a circumference of the inner surface, a plurality of ribs having a non-uniform pattern on the inner surface, a plurality of flat surfaces, a uniform or non-uniform roughened surface, a plurality of particulates or beads embedded in the sidewall, and any combinations thereof.

6. The rolling diaphragm syringe of claim 1, wherein the at least one surface texturization feature comprises a plurality of longitudinal ribs.

7. The rolling diaphragm syringe of claim 6, wherein the plurality of longitudinal ribs are uniformly or non-uniformly arranged circumferentially around the inner surface.

8. The rolling diaphragm syringe of claim 6, wherein the plurality of longitudinal ribs extend partially from the proximal end to the distal end.

9. The rolling diaphragm syringe of claim 6, wherein at least a portion of the plurality of longitudinal ribs extend for different lengths and at different regions along a longitudinal axis of the rolling diaphragm syringe.

10. The rolling diaphragm syringe of claim 6, wherein the plurality of longitudinal ribs have different heights along a length of a longitudinal axis of the rib.

11. (canceled)

12. The rolling diaphragm syringe of claim 1, wherein the at least one surface texturization feature maintains a fluid pathway between substantially parallel inner surfaces of the at least partially rolled rolling diaphragm syringe to allow air to escape from between the substantially parallel inner surfaces during a rolling or an unrolling process.

13. A preform for blow-molding a rolling diaphragm syringe, the preform comprising:
a closed proximal end portion having a piston engagement feature configured for releasable engagement between the rolling diaphragm syringe and a piston of a fluid injector;
a distal end having a fluid outlet;
a sidewall having an inner surface and an outer surface; and
at least one preform texturization feature on at least a portion of one or both of the inner surface and the outer surface of the sidewall, wherein the at least one preform texturization feature forms an at least one surface texturization feature on at least a portion of an inner surface of a sidewall of the rolling diaphragm syringe.

14. The preform of claim 13, wherein the at least one preform texturization feature comprises a plurality of preform longitudinal ribs on at least a portion of the inner surface of the sidewall, wherein the plurality of preform longitudinal ribs form a plurality of longitudinal ribs or a plurality of spiral ribs on at least a portion of the inner surface of the sidewall of the rolling diaphragm syringe.

15. The preform of claim 13, wherein the at least one preform texturization feature comprises a plurality of preform longitudinal ribs on at least a portion of the outer surface of the sidewall,
wherein the plurality of preform longitudinal ribs form a plurality of longitudinal ribs or a plurality of spiral ribs on at least a portion of an outer surface of the sidewall of the rolling diaphragm syringe, and
wherein the plurality of longitudinal ribs or the plurality of spiral ribs on at least a portion of the outer surface of the sidewall of the rolling diaphragm syringe are converted to a plurality of longitudinal ribs or a plurality of spiral ribs on at least a portion of the inner surface of the sidewall of the rolling diaphragm syringe during a blow-molding process.

16. The preform of claim 13, wherein the at least one preform texturization feature on at least the portion of one or both of the inner surface and the outer surface of the sidewall are formed during an injection molding process using a mold having corresponding grooves on an injection mold core structure or an outer injection mold cavity.

17. The preform of claim 13, the at least one preform texturization feature comprises a plurality of preform longitudinal ribs on at least a portion of the inner surface of the sidewall, wherein the plurality of preform longitudinal ribs are formed by etching the plurality of preform longitudinal grooves on the at least a portion of the inner surface of the sidewall during removing of an injection mold core structure having a corresponding plurality of groove etching members.

18. A method for reducing friction between contacting portions of a rolled inner surface of a flexible sidewall of a rolling diaphragm, the method comprising:
texturizing at least a portion of an inner surface of the flexible sidewall with at least one surface texturization feature selected from the group consisting of a plurality of uniform or non-uniform longitudinal ribs, a plurality of uniform or non-uniform ribs having a spiral configuration around a circumference of the inner surface, a plurality of ribs having a non-uniform pattern on the inner surface, a plurality of flat surfaces, a uniform or non-uniform roughened surface, a plurality of particulates or beads embedded in the flexible sidewall, and any combinations thereof.

19. The method of claim 18, wherein texturizing at least a portion of the inner surface comprises:
texturizing at least a portion of an inner surface of an injection molded preform; and
blow-molding the injection molded preform to provide the rolling diaphragm.

20. (canceled)

21. (canceled)

22. The method of claim 18, wherein reducing the friction between the contacting portions of the rolled inner surface of the flexible sidewall of the rolling diaphragm reduces or eliminates an audible squeak during a rolling or an unrolling of the rolling diaphragm.

23. The method of claim 18, wherein a coefficient of friction (μ) between contacting portions of the rolled inner surface of the flexible sidewall having the at least one surface texturization feature as the flexible sidewall is rolled or unrolled ranges from 0.10 to 2.30.

24. (canceled)

25. (canceled)
